(19) United States
(12) Patent Application Publication
(10) Pub. No.: US 2007/0111294 A1
(43) Pub. Date: May 17, 2007

(54) Methods and organisms for the growth-coupled production of succinate

(75) Inventors: Anthony P. Burgard, Bellefonte, PA (US); Stephen J. Van Dien, Solana Beach, CA (US)

Correspondence Address: MCDERMOTT, WILL & EMERY, 4370 LA JOLLA VILLAGE DRIVE, SUITE 700, SAN DIEGO, CA, 92122, US

(73) Assignee: Genomatica, Inc.

(21) Appl. No.: 11/518,502

(22) Filed: Sep. 8, 2006

Related U.S. Application Data
(60) Provisional application No. 60/715,723, filed on Sep. 9, 2005.

(51) Int. Cl. C12P 7/46; C12N 1/21; C12N 1/18; C12N 1/16; C12N 15/74

(52) U.S. Cl. 435/145; 435/471; 435/252.3; 435/254.2; 435/254.3; 435/484; 435/483

(57) ABSTRACT

The invention provides a non-naturally occurring microorganism comprising one or more gene disruptions encoding an enzyme associated with growth-coupled production of succinate when an activity of the enzyme is reduced, whereby the one or more gene disruptions confers stable growth-coupled production of succinate onto the non- naturally occurring microorganism. Also provided is a non-naturally occurring microorganism comprising a set of metabolic modifications obligatory coupling succinate production to growth of the microorganism, the set of metabolic modifications comprising disruption of one or more genes selected from the set of genes comprising: (a) adhE, ldhA; (b) adhE, ldhA, acka-pta; (c) pfl, ldhA; (d) pfl, ldhA, adhE; (e) acka-pta, pykF, atpF, sdhA; (f) acka-pta, pykF, ptsG, or (g) acka-pta, pykF, ptsG, adhE, ldhA, or an ortholog thereof, wherein the microorganism exhibits stable growth-coupled production of succinate. Additionally provided is a non-naturally occurring microorganism having the genes encoding the metabolic modification (e) acka-pta, pykF, atpF, sdhA that further includes disruption of at least one gene selected from pyka, atpH, sdhB or dhaKLM; a non-naturally occurring microorganism having the genes encoding the metabolic modification (f) ackA-pta, pykF, ptsG that further includes disruption of at least one gene selected from pykA or dhaKLM, or a non-naturally occurring microorganism having the genes encoding the metabolic modification (g) ackA-pta, pykF, ptsG, adhE, ldhA that further includes disruption of at least one gene selected from pykA or dhaKLM. The disruptions can be complete gene disruptions and the non-naturally occurring organisms can include a variety of prokaryotic or eukaryotic microorganisms. A method of producing a non-naturally occurring microorganism having stable growth-coupled production of succinate also is provided. The method includes: (a) identifying in silico a set of metabolic modifications requiring succinate production during exponential growth, and (b) genetically modifying a microorganism to contain the set of metabolic modifications requiring succinate production.

This application claims the benefit of priority of U.S. Provisional Application Ser. No. 60/715,723, filed Sep. 9, 2005, the entire contents of which is incorporated herein by reference.

This invention was made with government support under grant number 1 R43 GMO75531-01 awarded by the National Institutes of Health. The United States Government has certain rights in this invention.

### BACKGROUND OF THE INVENTION

This invention relates generally to in silico design of organisms and, more specifically to organisms having selected genotypes for the growth-coupled production of succinate.

Succinate is a compound of tremendous commercial interest due to its use as a precursor to commodity chemicals in the food, pharmaceutical, detergent and polymer industries. Fermentation-derived succinate could potentially supply over 2.7×10⁸ kg industrial products per year including 1,4 butanediol and related products, tetrahydrofuran, γ-butyrolactone, n-methyl pyrrolidinone (NMP) and 2-pyrrolidinone, Zeikus et al., Appl Microbiol Biotechnol, 51: 545-552 (1999). The basic chemistry of succinic acid is similar to that of the petrochemically-derived maleic acid/anhydride and thus only its production cost is preventing it from exploding into an expansive array of markets. Biological succinate production is also a green process where the greenhouse gas CO₂ must be fixed into succinate during sugar fermentation. For these reasons, the 1,4-diacids (succinate, fumarate, and malate) are among the set of twelve compounds identified by the Department of Energy as highest priority for the development of bioprocesses out of over 300 evaluated candidates.

Central metabolic compounds such as succinate provide good initial targets for metabolic engineering as they are often constitutively produced during basal metabolism. Examples of organisms innately capable of producing succinate from carbohydrates include Anaerobiospirillum succiniciproducens, Samuelov et al., Appl Environ Microbiol, 65: 2260-63 (1999), Lee et al., Appl Microbiol Biotechnol, 54: 23-27 (2000), Lee et al., Biotechnol Lett, 25: 111 - 14 (2003), Actinobacillus succinogenes, Guettler et al., Int J Syst Bacteriol, 49: 207-16 (1999), Urbance et al., Appl Microbiol Biotechnol, 65: 664-70 (2004), and the recently-sequenced bovine rumen bacterium, Mannheimia succiniciproducens, Lee et al., Bioprocess Biosyst Eng, 26: 63-7 (2003), Hong et al., Nat Biotechnol, 22: 1275-81 (2004), Lee et al., Appl Microbiol Biotechnol, 58: 663-8 (2002). In addition, some reports have purported to achieve the construction of Escherichia coli strains with improved succinate yields through various metabolic engineering strategies. These efforts have focused on increasing the channeling of carbon flux toward succinate and the availability of the cofactor NADH. For example, the overexpression of PEP carboxylase (ppc), Millard et al., Appl Environ Microbiol, 62: 1808-10 (1996), and the expression of Rhizobium etli pyruvate carboxylase (pyc), Gokarn et al., Biotechnol Lett, 20: 795-798 (1998), have led to succinate yields in E. coli of 0.30 g/g and 0.17 g/g, respectively, by increasing the flux into the succinate branch of the TCA cycle. In addition, E. coli mutants deficient in lactate dehydrogenase (ldh) and pyruvate formate lyase (pfl) (i.e., strain NZN 111), in conjunction with the overexpression of the E. coli, Stols et al., Appl Environ Microbiol, 63: 2695-701 (1997), Hong et al., Biotechnol Bioeng, 74: 89-95 (2001), or Ascaris suum, Stols et al., Appl Biochem Biotechnol, 63-65: 153-8 (1997), malic enzyme, have achieved improved succinate yields. An additional spontaneous chromosomal mutation in NZN 111, later mapped to the ptsG gene of the phosphotransferase system, Chatterjee et al., Appl Environ Microbiol, 67: 148-54 (2001), led to strain AFP111 with an anaerobic succinate yield of 1 mol/mol glucose (0.66 g/g), Donnelly et al., Appl Biochem Biotechnol, 70-72: 187-98 (1998). Various properties of strains NZN111 and AFP111 in the presence and absence of the R. etli pyruvate carboxylase have been investigated under anaerobic and dual-phase conditions (i.e., aerobic growth followed by anaerobic production), Vemuri et al., Appl Environ Microbiol, 68: 1715-27, 18 (2002). Vemuri et al., J Ind Microbiol Biotechnol, 28: 325-32 (2002), resulting in yields of about 0.96 g/g. Other efforts have resulted in succinate-producing strains of E. coli capable of achieving 0.91 mol/mol (0.60 g/g) aerobically, Lin et al., Metab Eng, (2005). In Press, and 1.6 mol/mol (1.0 g/g) anaerobically, Sanchez et al., Metab Eng, 7: 229-39 (2005).

However, despite the above efforts and reports purporting the development of certain bacterial strains producing increased succinate yields, the approaches employed have several drawbacks which hinder applicability in commercial settings. As described further below, the stains produced by the above methods generally are unstable in commercial fermentation processes due to selective pressures favoring the unaltered or wild-type parental counterparts.

Thus, there exists a need for microorganisms having commercially beneficial characteristics that obligatory link biosynthesis of a desired product to optimal culture conditions. The present invention satisfies this need and provides related advantages as well.

### SUMMARY OF THE INVENTION

The invention provides a non-naturally occurring microorganism comprising one or more gene disruptions encoding an enzyme associated with growth-coupled production of succinate when an activity of the enzyme is reduced, whereby the one or more gene disruptions confers stable growth-coupled production of succinate onto the non- naturally occurring microorganism. Also provided is a non-naturally occurring microorganism comprising a set of metabolic modifications obligatory coupling succinate production to growth of the microorganism, the set of metabolic modifications comprising disruption of one or more genes selected from the set of genes comprising: (a) adhE, ldhA; (b) adhE, ldhA, acka-pta; (c)pfl, IdhA; (d) pfi, ldhA, adhE; (e) ackA-pta, pykF, atpF, sdhA; (f) ackA-pta, pykF, ptsG, or (g) acka-pta, pykF, ptsG, adhE, ldhA, or an ortholog thereof, wherein the microorganism exhibits stable growth-coupled production of succinate. Additionally provided is a non-naturally occurring microorganism having the genes encoding the metabolic modification (e) ackA-pta, pykF, atpF, sdhA that further includes disruption of at least one gene selected from pykA, atpH, sdhB or dhaKLM; a non-naturally occurring microorganism having the genes encoding the metabolic modification (f) ackA-pta, pykF, ptsG that further includes disruption of at least one gene selected from pykA or dhaKLM, or a non-naturally occurring microorganism having the genes encoding the metabolic modification (g) ackA-pta, pykF, ptsG, adhE, ldhA that further includes disruption of at least one gene selected from pykA or dhaKLM. The disruptions can be complete gene disruptions and the non-naturally occurring organisms can include a variety of prokaryotic or eukaryotic microorganisms. A method of producing a non-naturally occurring microorganism having stable growth-coupled production of succinate also is provided. The method includes: (a) identifying in silico a set of metabolic modifications requiring succinate production during exponential growth, and (b) genetically modifying a microorganism to contain the set of metabolic modifications requiring succinate production.

### BRIEF DESCRIPTION OF THE DRAWINGS

FIG. 1 shows an estimated depiction of the tradeoff between biochemical production and cell growth. Points A and B represent the maximum biomass solution of the wild-type and mutant strains, respectively. Note that the mutant strain exhibits growth-coupled production.

FIG. 2 shows a bilevel optimization structure of OptKnock. The inner problem performs the flux allocation based on the optimization of a particular cellular objective. The outer problem then maximizes the bioengineering objective (e.g., compound overproduction) by restricting access to key reactions available to the optimization of the inner problem.

FIG. 3 shows succinate versus growth rate boundaries for four mutant strains as compared to the wild-type. Completely anaerobic conditions are assumed along with a basis glucose uptake rate of 10 mmol/(gDW-hr). All strains follow the outer boundary, which also corresponds to the wild type strain, but diverge downward from the left as follows: adhE, ldh, pta (first divergence, red); pfl ldh, adhE (second divergence, green); adhE, ldh (third divergence, blue), and pfl, ldh (fourth divergence, grey).

FIG. 4 shows succinate versus growth rate boundaries for the OptKnock-derived mutant strain (ackA-pta, atpFH, pykA, pykF, dhaKLM, sdhAB) compared to the wild-type. A basis glucose uptake rate of 10 mmol/(gDW-hr) is assumed. Strains correspond to the lines starting from the left as follow: ackA-pta, atpFH, pykA, pykF, dhaKLM, sdhAB (anaerobic) (fisrt line, green); ackA-pta, atpFH, pykA, pykF, dhaKLM, sdhAB (aerobic) (second line, red); wild-type (anaerobic) (third line, blue), and wild-type (aerobic) (fourth line, black)

FIG. 5 shows succinate versus growth rate boundaries for the OptKnock-derived mutant strain (ackA-pta, pykA, pykF, ptsG, dhaKLM) compared to the wild-type for various non-growth associated ATP maintenance requirements. A basis glucose uptake rate of 10 mmol/(gDW-hr) is assumed. Strains correspond to the lines starting from the left as follow: ackA-pta, pykA, pykF, ptsG, dhaKLM (ATPM=6) (outer boundary intersection on y-axisis, green); ackA-pta, pykA, pykF, ptsG, dhaKLM (ATPM=3) (second line, red); ackA-pta, pykA, pykF, ptsG, dhaKLM (ATPM=0) (third line, blue), and wild-type (outer boundary, black).

FIG. 6 shows succinate versus growth rate boundaries for the OptKnock-derived mutant strain (ackA-pta, pykA, pykF, ptsG, dhaKLM) at various oxygenation rates. The typical 7.6 mmol/(gDW-hr) maintenance energy requirement is imposed. A basis glucose uptake rate of 10 mmol/(gDW-hr) is assumed. Strains correspond to the lines starting from the left as follow: ackA-pta, pykA, pykF, ptsG, dhaKLM (O2<2) (first line, green); ackA-pta, pykA, pykF, ptsG, dhaKLM (O2<5) (second line, red); ackA-pta, pykA, pykF, ptsG, dhaKLM (O2<10) (third line, blue), and pta, ptsG, f6pa, pyk (O2 unlimited) (outer boundary, black).

FIG. 7 shows succinate versus growth rate boundaries for the OptKnock-derived mutant strain (ackA-pta, pykA, pykF, ptsG, dhaKLM, ldh, adhE) at various oxygenation rates. The typical 7.6 mmol/(gDW-hr) maintenance energy requirement is imposed. A basis glucose uptake rate of 10 mmol/(gDW-hr) is assumed. Strains correspond to the lines starting from the left as follow: ackA-pta, pykA, pykF, ptsG, dhaKLM, adhE, ldh (O2<2) (first line, green); ackA-pta, pykA, pykF, ptsG, dhaKLM, adhE, ldh (O2<5) (second line, red); ackA-pta, pykA, pykF, ptsG, dhaKLM, adhE, ldh (O2<10) (third line, blue), and ackA-pta, pykA, pykF, ptsG, dhaKLM, adhE, ldh (O2 unlimited) (outer boundary, black).

FIG. 8 shows the performance of the OptKnock-designed succinate strains pre-evolution. (a) Strain construction strategy. AB1 and AB2 are intermediate strains, which were also characterized to evaluate the effect of the final knockout in each strain lineage. (b) Mass succinate yield of each strain over the entire experiment averaged over three independent cultures.

FIG. 9 shows the approximate doubling times, determined from the frequency of dilutions, of strains MG 1655 and AB3, plotted throughout the course of the evolutions.

FIG. 10 shows the product profile of samples taken from the Evolugator during evolution of strain AB3. Diamonds (blue), succinate; Triangles (red), formate; Circles (green), acetate.

FIG. 11 shows the mass (a) and molar (b) yields of succinate and other fermentation products present after the microaerobic culturing of E. coli strains, relative to total glucose metabolized. Left bars, wild-type MG1655; middle bars, unevolved AB3; right bars, evolved AB3

FIG. 12 shows the mass percentages of fermentation products present after the microaerobic culturing of the pre-evolved (left bars) and post-evolved (right bars) AB3 strain are shown.

### DETAILED DESCRIPTION OF THE INVENTION

This invention is directed to the design and production of cells and organisms having growth-coupled production of succinate. In one embodiment, the invention utilizes optimization-based approaches based on in silico stoichiometric models of Escherichia coli metabolism that identify metabolic designs for optimal production of succinate. A bilevel programming framework, OptKnock, is applied within an iterative algorithm to predict multiple sets of gene disruptions, that collectively result in the growth-coupled production of succinate. The results described herein indicate that combinations of strategically placed gene deletions or functional disruptions of genes significantly improves the succinate production capabilities of Escherichia coli and other cells or organisms. Growth-coupled production of succinate for the in silico designs are confirmed by construction of strains having the designed metabolic genotype. These metabolically engineered cells or organisms also can be subjected to adaptive evolution to further augment growth-coupled succinate production.

In a further embodiment, the invention is directed to an integrated computational and engineering platform for developing metabolically altered microorganism strains having enhanced succinate producing characteristics. Strains identified via the computational component of the platform are put into actual production by genetically engineering the predicted metabolic alterations which lead to the enhanced production of succinate. Production of succinate is coupled to optimal growth of the microorganism to optimize yields of this product during fermentation. In yet a further embodiment, strains exhibiting growth-coupled production of succinate are further subjected to adaptive evolution to further augment product biosynthesis. The levels of growth-coupled succinate production following adaptive evolution also can be predicted by the computational component of the system where, in this specific embodiment, the elevated succinate levels are realized only following evolution.

As used herein, the term “non-naturally occurring” when used in reference to a microorganism of the invention is intended to mean that the microorganism has at least one genetic alteration not normally found in a wild-type strain of the referenced species. The genetic alteration can be a gene deletion or some other functional disruption of the genetic material.

As used herein, the term “microorganism” is intended to mean a prokaryotic or eukaryotic cell or organism having a microscopic size. The term is intended to include bacteria of all species and eukaryotic organisms such as yeast and fungi. The term also includes cell cultures of any species that can be cultured for the production of a biochemical.

As used herein, the term “growth-coupled” when used in reference to the production of a biochemical is intended to mean that the biosynthesis of the referenced biochemical is an obligatory product produced during the growth phase of a microorganism.

As used herein, the term “metabolic modification” is intended to refer to a biochemical reaction that is altered from its naturally occurring state. Metabolic modifications can include, for example, elimination of a biochemical reaction activity by functional disruptions of one or more genes encoding an enzyme participating in the reaction. Sets of exemplary metabolic modifications are illustrated in Table 1. Individual reactions specified by such metabolic modifications and their corresponding gene complements are exemplified in Table 2 for E. coli. Reactants and products utilized in these reactions are exemplified in Table 3.

As used herein, the term “succinate” is intended to mean the dicarboxylic acid HOOCCH₂CH₂COOH that is formed in the Krebs cycle and in various fermentation processes. The term “succinate” as it is used herein is synonymous with the term “succinic acid.” Chemically, succinate corresponds to a salt or ester of succinic acid. Therefore, succinate and succinic acid refer to the same compound, which can be present in either of the two forms depending on the pH of the solution.

As used herein, the term “gene disruption,” or grammatical equivalents thereof, is intended to mean a genetic alteration that renders the encoded gene product inactive. The genetic alteration can be, for example, deletion of the entire gene, deletion of a regulatory sequence required for transcription or translation, deletion of a portion of the gene with results in a truncated gene product or by any of various mutation strategies that inactivate the encoded gene product. One particularly useful method of gene disruption is complete gene deletion because it reduces or eliminates the occurrence of genetic reversions in the non-naturally occurring microorganisms of the invention.

As used herein, the term “stable” when used in reference to growth-coupled production of a biochemical product is intended to refer to microorganism that can be cultured for greater than five generations without loss of the coupling between growth and biochemical synthesis. Generally, stable growth-coupled biochemical production will be greater than I 0 generations, particularly stable growth-coupled biochemical production will be greater than about 25 generations, and more particularly, stable growth-coupled biochemical production will be greater than 50 generations, including indefinitely. Stable growth-coupled production of a biochemical can be achieved, for example, by deletion of a gene encoding an enzyme catalyzing each reaction within a set of metabolic modifications. The stability of growth-coupled production of a biochemical can be enhanced through multiple deletions, significantly reducing the likelihood of multiple compensatory reversions occurring for each disrupted activity.

Those skilled in the art will understand that the metabolic modifications exemplified herein are described with reference to E. coli genes and their corresponding metabolic reactions. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous gene disruptions in the other species. Such disruptions can include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the growth-coupled production of a biochemical product, those skilled in the art will understand that the orthologous gene harboring the metabolic activity to be disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase 11) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene compared to a gene encoding the function sought to be substituted. Therefore, a nonorthologous gene includes, for example, a paralog or an unrelated gene.

Therefore, in identifying and constructing the non-naturally occurring microorganisms of the invention having growth-coupled production of a biochemical, those skilled in the art will understand with applying the teaching and guidance provided herein to a particular species that the identification of metabolic modifications should include identification and disruption of orthologs. To the extent that paralogs and/or nonorthologous gene displacements are present in the referenced microorganism that encode an enzyme catalyzing a similar or substantially similar metabolic reaction, those skilled in the art also can eliminate these evolutionally related genes to ensure that any functional redundancy in enzymatic activities do not short circuit the designed metabolic modifications.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compared and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarly to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 05, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: -2; gap open: 5; gap extension: 2; x_dropoff: 50; expect: 10.0; wordsize: I 1; filter: off. Those skilled in the art will know what modifications can be made to the above parameters to either increase or decrease the stringency of the comparison, for example, and determine the relatedness of two or more sequences.

The invention provides a method of producing a non-naturally occurring microorganism having stable growth-coupled production of succinate. The method includes: (a) identifying in silico a set of metabolic modifications requiring succinate production during cell growth, and (b) genetically modifying a microorganism to contain the set of metabolic modifications requiring succinate production.

An important consideration for bioprocessing is whether to use a batch or continuous fermentation scheme. One difference between the two schemes that will influence the amount of product produced is the presence of a preparation, lag, and stationary phase for the batch scheme in addition to the exponential growth phase. In contrast, continuous processes are kept in a state of constant exponential growth and, if properly operated, can run for many months at a time. For growth-associated and mixed-growth-associated product formation, continuous processes provide much higher productivities (i.e., dilution rate times cell mass) due to the elimination of the preparation, lag, and stationary phases. For example, given the following reasonable assumptions:

Monod kinetics (i.e., μ=μₘ·S/(Kₛ+S))

μₘ=1.0 hr⁻¹

final cell concentration/initial cell concentration=20

Tₚᵣₑₚ+t_{log}+tₛₜₐₜ=5 hr

feed concentration of limiting nutrient >>Ks

increased productivity from a continuous process has been estimated at 8-fold, Shuler et al, Prentice Hall, Inc.: Upper Saddle River, N.J., 245-247.

Despite the overwhelming advantage in productivity, many more batch processes are in operation than continuous processes for a number of reasons. First, for non-growth associated product formation (e.g., penicillin), the productivity of a batch system may significantly exceed that of a continuous process because the latter would have to operate at very low dilution rates. Next, production strains generally have undergone modifications to their genetic material to improve their biochemical or protein production capabilities. These specialized strains are likely to grow less rapidly than their parental complements whereas continuous processes such those employing chemostats (fermenters operated in continuous mode) impose large selection pressures for the fastest growing cells. Cells containing recombinant DNA or carrying point mutations leading to the desired overproduction phenotype are susceptible to back-mutation into the original less productive parental strain. It also is possible for strains having single gene deletions to develop compensatory mutations that will tend to restore the wild-type growth phenotype. The faster growing cells usually out-compete their more productive counterparts for limiting nutrients, drastically reducing productivity. Batch processes, on the other hand, limit the number of generations available by not reusing cells at the end of each cycle, thus decreasing the probability of the production strain reverting back to its wild-type phenotype. Finally, continuous processes are more difficult to operate long-term due to potential engineering obstacles such as equipment failure and foreign organism contamination. The consequences of such failures also are much more considerable for a continuous process than with a batch culture.

For small-volume production of specialty chemicals and/or proteins, the productivity increases of continuous processes rarely outweigh the risks associated with strain stability and reliability. However, for the production of large-volume, growth-associated products such as succinate, the increases in productivity for a continuous process can result in significant economic gains when compared to a batch process. Although the engineering obstacles associated with continuous bioprocess operation would always be present, the strain stability concerns can be overcome through metabolic engineering strategies that reroute metabolic pathways to reduce or avoid negative selective pressures and favor production of the target product during the exponential growth phase.

One computational method for identifying and designing metabolic alterations favoring growth-coupled production of a product is the OptKnock computational framework, Burgard et al., Biotechnol Bioeng, 84: 647-57 (2003). OptKnock is a metabolic modeling and simulation program that suggests gene deletion strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that enable an effective query of the performance limits of metabolic networks and provides methods for solving the resulting mixed-integer linear programming problems. The metabolic modeling and simulation methods referred to herein as OptKnock are described in, for example, U.S. Patent Application Ser. No.10/043,440, filed Jan. 10, 2002, and in International Patent No. PCT/US02/00660, filed Jan. 10, 2002.

Another computational method for identifying and designing metabolic alterations favoring growth-coupled production of a product is metabolic modeling and simulation system termed SimPheny®. This computational method and system is described in, for example, U.S. Patent Application Ser. No. 10/173,547, filed Jun. 14, 2002, and in International Patent Application No. PCT/US03/18838, filed Jun. 13,2003.

SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components. Analysis methods such as convex analysis, linear programming and the calculation of extreme pathways as described, for example, in Schilling et al., J. Theor. Biol. 203:229-248 (2000); Schilling et al., Biotech. Bioeng. 71:286-306 (2000) and Schilling et al., Biotech. Prog. 15:288-295 (1999), can be used to determine such phenotypic capabilities. [046] As described above, one constraints-based method used in the computational programs applicable to the invention is flux balance analysis. Flux balance analysis is based on flux balancing in a steady state condition and can be performed as described in, for example, Varma and Palsson, Biotech. Bioeng. 12:994-998 (1994). Flux balance approaches have been applied to reaction networks to simulate or predict systemic properties of, for example, adipocyte metabolism as described in Fell and Small, J. Biochem. 138:781-786 (1986), acetate secretion from E. coli under ATP maximization conditions as described in Majewski and Domach, Biotech. Bioeng. 35:732-738 (1990) or ethanol secretion by yeast as described in Vanrolleghem et al., Biotech. Prog. 12:434-448 (1996). Additionally, this approach can be used to predict or simulate the growth of E. coli on a variety of single-carbon sources as well as the metabolism of H. influenzae as described in Edwards and Palsson, Proc. Natl. Acad. Sci. 97:5528-5533 (2000), Edwards and Palsson, J. Bio. Chem. 274:17410-17416 (1999) and Edwards et al., Nature Biotech. 19:125-130 (2001).

Once the solution space has been defined, it can be analyzed to determine possible solutions under various conditions. This computational approach is consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement growth-coupled production of a biochemical product. Such metabolic modeling and simulation methods include, for example, the computational systems exemplified above as SimPheny® and OptKnock. For simplicity in illustrating the invention, the methods and strains will be described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

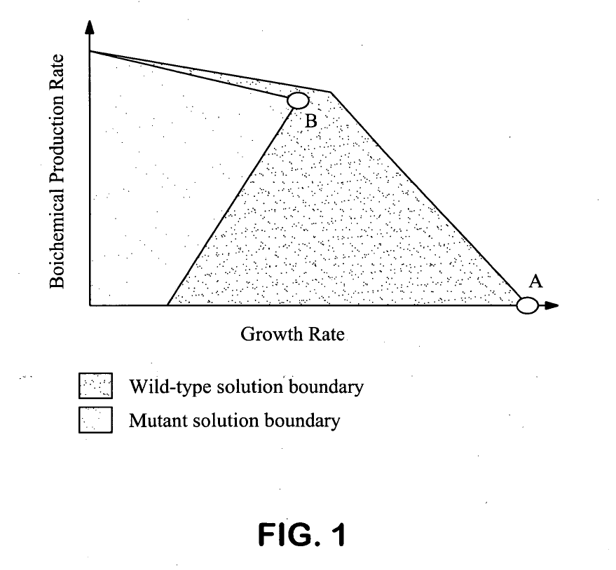

The ability of a cell or organism to obligatory couple growth to the production of a biochemical product can be illustrated in the context of the biochemical production limits of a typical metabolic network calculated using an in silico model. These limits are obtained by fixing the uptake rate(s) of the limiting substrate(s) to their experimentally measured value(s) and calculating the maximum and minimum rates of biochemical production at each attainable level of growth. As shown in FIG. 1, the production of a desired biochemical generally is in direct competition with biomass formation for intracellular resources. Under these circumstances, enhanced rates of biochemical production will necessarily result in sub-maximal growth rates. The knockouts suggested by the above metabolic modeling and simulation programs such as OptKnock are designed to restrict the allowable solution boundaries forcing a change in metabolic behavior from the wild-type strain as depicted in FIG. 1. Although the actual solution boundaries for a given strain will expand or contract as the substrate uptake rate(s) increase or decrease, each experimental point will lie within its calculated solution boundary. Plots such as these enable accurate predictions of how close the designed strains are to their performance limits which also indicates how much room is available for improvement.

The OptKnock mathematical framework is exemplified herein for pinpointing gene deletions leading to growth-coupled biochemical production as illustrated in FIG. 1. The procedure builds upon constraint-based metabolic modeling which narrows the range of possible phenotypes that a cellular system can display through the successive imposition of governing physico-chemical constraints, Price et al., Nat Rev Microbiol, 2: 886-97 (2004). As described above, constraint-based models and simulations are well known in the art and generally invoke the optimization of a particular cellular objective, subject to network stoichiometry, to suggest a likely flux distribution.

Briefly, the maximization of a cellular objective quantified as an aggregate reaction flux for a steady state metabolic network comprising a set N={1, . . . , N} of metabolites and a set M={1, . . . , M} of metabolic reactions is expressed mathematically as follows:

$$maximize {v}_{cellular objective}$$

$$subject to {\sum }_{j = 1}^{M} {S}_{ij} {v}_{j} = 0 , ∀ i ∈ N$$

$${v}_{substrate} = {v}_{substrate\_uptake} mmol \text{/} gDW \cdot hr ∀ i ∈ \{ limiting substrate ( s ) \}$$

$${v}_{atp} \geq {v}_{atp\_main} mmol \text{/} gDW \cdot hr$$

$${v}_{j} \geq 0 , ∀ j ∈ \{ irrev . reactions \}$$

where Sᵢⱼ is the stoichiometric coefficient of metabolite i in reactionj, vⱼ is the flux of reactionj, V_{substrate—uptake} represents the assumed or measured uptake rate(s) of the limiting substrate(s), and v_{atp—main} is the non-growth associated ATP maintenance requirement. The vector v includes both internal and external fluxes. In this study, the cellular objective is often assumed to be a drain of biosynthetic precursors in the ratios required for biomass formation, Neidhardt, F.C. et al., 2nd ed. 1996, Washington, D.C.: ASM Press. 2 v. (xx, 2822, lxxvi ). The fluxes are generally reported per 1gD W·hr (gram of dry weight times hour) such that biomass formation is expressed as g biomass produced/gDW·hr or 1/hr.

The modeling of gene deletions, and thus reaction elimination, first employs the incorporation of binary variables into the constraint-based approach framework, Burgard et al., Biotechnol Bioeng, 74: 364-375 (2001), Burgard et al., Biotechnol Prog, 17: 791-797 (2001). These binary variables,

$${y}_{j} = \{ \begin{matrix}1 , if reaction flux {v}_{j} is active \\ 0 , if reaction flux {v}_{j} is not active\end{matrix} , ∀ j ∈ M$$

assume a value of 1 if reaction j is active and a value of 0 if it is inactive. The following constraint,

### vⱼ^{min}·yⱼ≦vⱼ≦vⱼ^{max}·yⱼ, ∀jεM

ensures that reaction flux vⱼ is set to zero only if variable yⱼ is equal to zero. Alternatively, when yⱼ is equal to one, vⱼ is free to assume any value between a lower vⱼ^{min} and an upper vⱼ^{max} bound. Here, vⱼ^{min} and vⱼ^{max} are identified by minimizing and maximizing, respectively, every reaction flux subject to the network constraints described above, Mahadevan et al., Metab Eng, 5: 264-76 (2003).

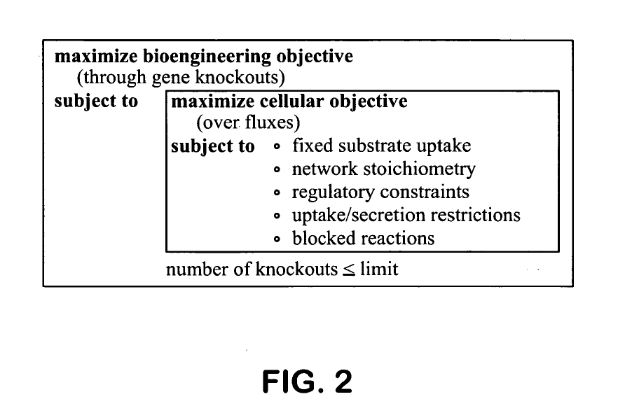

Optimal gene/reaction knockouts are identified by solving a bilevel optimization problem that chooses the set of active reactions (yⱼ=1) such that an optimal growth solution for the resulting network overproduces the chemical of interest. Schematically, this bilevel optimization problem is illustrated in FIG. 2. Mathematically, this bilevel optimization problem is expressed as the following bilevel mixed-integer optimization problem:

$${maximize}_{{y}_{j}} {v}_{chemical} ( OptKnock )$$

$$( \begin{matrix}{subject}_{{v}_{j}} to maximize {v}_{biomass} \\ subject to {\sum }_{j = 1}^{M} {S}_{ij} {v}_{j} = 0 , ∀ i ∈ N \\ {v}_{substrate} = {v}_{substrate\_uptake} ∀ i ∈ \{ limiting substrate ( s ) \} \\ {v}_{atp} \geq {v}_{atp\_main}\end{matrix} )$$

$${v}_{biomass} \geq {v}_{biomass}^{target}$$

$${v}_{j}^{min} \cdot {y}_{j} \leq {v}_{j} \leq {v}_{j}^{max} \cdot {y}_{j} , ∀ j ∈ M$$

$${\sum }_{j ∈ {M}^{forward}} ( 1 - {y}_{j} ) = K$$

$${y}_{j} ∈ \{ 0 , 1 \} , ∀ j ∈ M$$

where V_{chemical} is the production of the desired target product, for example succinate or other biochemical product, and K is the number of allowable knockouts. Note that setting K equal to zero returns the maximum biomass solution of the complete network, while setting K equal to one identifies the single gene/reaction knockout (yⱼ=0) such that the resulting network involves the maximum overproduction given its maximum biomass yield. The final constraint ensures that the resulting network meets a minimum biomass yield. Burgard et al., Biotechnol Bioeng, 84: 647-57 (2003), provide a more detailed description of the model formulation and solution procedure. Problems containing hundreds of binary variables can be solved in the order of minutes to hours using CPLEX 8.0, GAMS: The Solver Manuals. 2003: GAMS Development Corporation, accessed via the GAMS, Brooke et al., GAMS Development Corporation (1998), modeling environment on an IBM RS6000-270 workstation. The OptKnock framework has already been able to identify promising gene deletion strategies for biochemical overproduction, Burgard et al., Biotechnol Bioeng, 84: 647-57 (2003), Pharkya et al., Biotechnol Bioeng, 84: 887-899 (2003), and establishes a systematic framework that will naturally encompass future improvements in metabolic and regulatory modeling frameworks.

Any solution of the above described bilevel OptKnock problem will provide one set of metabolic reactions to disrupt. Elimination of each reaction within the set or metabolic modification can result in succinate as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve growth-coupled succinate production are implemented in the target cell or organism by functional disruption of at least one gene encoding each metabolic reaction within the set. As described previously, one particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the succinate coupling are desired or when genetic reversion is less likely to occur.

To identify additional productive solutions to the above described bilevel OptKnock problem which lead to further sets of reactions to disrupt or metabolic modifications that can result in the growth-coupled production of succinate or other biochemical products, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatory couples product biosynthesis to growth. For example, if a previously identified growth-coupled metabolic modification specifies reactions 1, 2, and 3 for disruption, then the following constraint prevents the same reactions from being simultaneously considered in subsequent solutions: y₁+Y₂+Y₃≧1. The integer cut method is well known in the art and can be found described in, for example, reference, Burgard et al., Biotechnol Prog, 17: 791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny.

Constraints of the above form preclude identification of larger reaction sets that include previously identified sets. For example, employing the integer cut optimization method above in a further iteration would preclude identifying a quadruple reaction set that specified reactions 1, 2, and 3 for disruption since these reactions had been previously identified. To ensure identification of all possible reaction sets leading to growth-coupled production of a product, a modification of the integer cut method was employed.

Briefly, the modified integer cut procedure begins with iteration ‘zero’ which calculates the maximum production of the desired biochemical at optimal growth for a wild-type network. This calculation corresponds to an OptKnock solution with K equaling 0. Next, single knockouts are considered and the two parameter sets, objstoreᵢₜₑᵣ and ystoreᵢₜₑᵣⱼ, are introduced to store the objective function (V_{chemical}) and reaction on-off information (yⱼ), respectively, at each iteration, iter. The following constraints are then successively added to the OptKnock formulation at each iteration.

$${v}_{chemical} \geq {objstore}_{iter} + ɛ - M \cdot {\sum }_{j ∈ {ystore}_{iter \cdot j} = 0} {y}_{j}$$

In the above equation, ε and M are a small and a large numbers, respectively. In general, ε can be set at about 0.01 and M can be set at about 1000. However, numbers smaller and/or larger then these numbers also can be used. M ensures that the constraint can be binding only for previously identified knockout strategies, while ε ensures that adding knockouts to a previously identified strategy must lead to an increase of at least ε in biochemical production at optimal growth. The approach moves onto double deletions whenever a single deletion strategy fails to improve upon the wild-type strain. Triple deletions are then considered when no double deletion strategy improves upon the wild-type strain, and so on. The end result is a ranked list, represented as desired biochemical production at optimal growth, of distinct deletion strategies that differ from each other by at least one knockout. This optimization procedure as well as the identification of a wide variety of reaction sets that, when disrupted, lead to the growth-coupled production of a biochemical product are exemplified in detail further below in the Examples. The Examples further exemplify the growth-coupled production of succinate. However, given the teachings and guidance provided herein, those skilled in the art will understand that the methods and metabolic engineering designs exemplified herein are equally applicable to the obligatory coupling of cell or microorganism growth to any biochemical product.

Employing the methods exemplified above and further illustrated in the Examples below, the methods of the invention enable the construction of cells and organisms that obligatory couple the production of a target biochemical product to growth of the cell or organism engineered to harbor the identified genetic alterations. In this regard, metabolic alterations have been identified that obligatory couple the production of succinate to microorganism growth. Microorganism strains constructed with the identified metabolic alterations produce elevated levels of succinate during the exponential growth phase. These strains can be beneficially used for the commercial production of succinate in continuous fermentation process without being subjected to the negative selective pressures described previously.

Therefore, the methods of the invention provide a set of metabolic modifications that are identified by an in silico method selected from OptKnock or SimPheny. The set of metabolic modifications can include functional disruption of one or more metabolic reactions including, for example, disruption by gene deletion. The metabolic modifications can be selected from the set of metabolic modifications listed in Table 1.

Also provided is a method of producing a non-naturally occurring microorganism having stable growth-coupled production of succinate. The method includes: (a) identifying in silico a set of metabolic modifications requiring succinate production during exponential growth; (b) genetically modifying a microorganism to contain the set of metabolic modifications requiring succinate production, and culturing the genetically modified microorganism. Culturing can include adaptively evolving the genetically modified microorganism under conditions requiring succinate production. The methods of the invention are applicable to bacterium, yeast and fungus as well as a variety of other cells and microorganism. The bacteria can include, for example, E. coli, A. succiniciproducens, A. succinogenes, M. succiniciproducens and R. etli.

A microorganism produced by the methods of the invention is further provided. Additionally, the invention provides non-naturally occurring microorganism comprising one or more gene disruptions encoding an enzyme associated with growth-coupled production of succinate and exhibiting stable growth-coupled production of succinate. The non-naturally occurring microorganisms of the invention include one or more gene disruptions occurring in genes encoding an enzyme obligatory coupling succinate production to growth of the microorganism when the gene disruption reduces an activity of the enzyme, whereby the one or more gene disruptions confers stable growth-coupled production of succinate onto the non-naturally occurring microorganism.

The non-naturally occurring microorganism can have one or more gene disruptions included in a metabolic modification listed in Table 1. The one or more gene disruptions can be a deletion. The non-naturally occurring microorganism of the invention can be selected from the group of microorganisms having a metabolic modification listed in Table 1. Non-naturally occurring microorganisms of the invention include bacteria, yeast, fungus or any of a variety of other microorganisms applicable to fermentation processes.Exemplary bacteria include species selected from E. coli, A. succiniciproducens, A. succinogenes, M. succiniciproducens, R. etli, Bacillus subtilis, Corynebacterium glutamicurn, Gluconobacter oxydans, Zymomonas mobilis, Lactococcus lactis, Lactobacillis plantarum, Streptomyces coelicolor, Clostridium acetobutylicum, Pseudomonas.fluiorescens, and Pseudomonas putida. Exemplary yeasts include species selected from Saccharomyces cerevisiae, Schizosaccharomyces pombe, Kluyveromyces lactis, Kluyveromyces marxianus, Aspergillus terreus, Aspergillus niger and Pichia pastoris.

The microorganisms having growth-coupled succinate production are exemplified herein with reference to an E. coli genetic background. However, with the complete genome sequence available for now more than 550 species (with more than half of these available on public databases such as the NCBI), including 395 microorganism genomes and a variety of yeast, fungi, plant, and mammalian genomes, the identification of an alternate species homolog for one or more genes, including for example, orthologs, paralogs and nonorthologous gene displacements, and the interchange of genetic alterations between organisms is routine and well known in the art. Accordingly, the metabolic alterations enabling growth-coupled production of succinate described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

For example, succinate production can be coupled to exponential growth in E. coli by deletion or functional removal of one or more genes encoding enzymes catalyzing the reaction referred to herein as ADHEr and one or more genes encoding enzymes catalyzing the reaction referred to herein as LDH_D. As shown in Table 2, an E. coli gene that encodes an enzyme catalyzing the adhE reaction is adhE or b 1241. Also, shown in Table 2 are two E. coli genes that encode an enzyme catalyzing the LDH_D reaction. These two LDH_D associated genes are b2133 and b1380. The common name for b1380 is ldhA. Theb2133 geneis an orthologof ldhA. To produce a metabolically engineered E. coli exhibiting growth coupled succinate production, genes encoding at least one enzyme catalyzing each of the ADHEr and LDH_D reactions have to be functionally disrupted. Such a disruption can occur, for example, by deleting the b1241 gene (adhE) and any of the b1380 gene (ldhA), its ortholog b2133 or both b1241 and b2133. For the growth-coupled production of succinate in a cell or organism other then E. coli the genes encoding comparable reactions for ADHEr and LDH_D in the species of interest can be functionally disrupted. For those organisms having analogous metabolic pathways such disruption can be accomplished by deleting, for example, the species homologue to b1241 and either of the b2133 or b1380 genes. As described previously, such homologues can include othologs and/or nonorthologous gene displacements. In some instances, such as when a substitute metabolic pathway exists in the species of interest, functional disruption can be accomplished by, for example, deletion of a paralog that catalyzes a similar, yet non-identical metabolic reaction which replaces the referenced reaction. Because certain differences among metabolic networks between different organisms, those skilled in the art will understand that the actual genes disrupted between different organisms may differ. However, the given the teachings and guidance provided herein, those skilled in the art also will understand that the methods of the invention can be applied to all microorganisms to identify the cognate metabolic alterations between organisms and to construct an organism in a species of interest that will enhance the coupling of succinate biosynthesis to growth.

The invention will be described herein with general reference to the metabolic reaction, reactant or product thereof, or with specific reference to one or more genes associated with the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction and that reference to any of these metabolic constitutes also references the gene or genes encoding the enzymes that catalyze the referenced reaction, reactant or product. Likewise, given the well known fields of metabolic biochemistry, enzymology and genomics, reference herein to a gene also constitutes a reference to the corresponding encoded enzyme and the reaction it catalyzes as well as the reactants and products of the reaction. As described previously and further below, exemplary reactions, reaction nomenclature, reactants, products, cofactors and genes encoding enzymes catalyzing a reaction involved in the growth-coupled production of succinate are set forth in Tables 1, 2 and 3.

The invention provides microorganisms having growth-coupled production of succinate. Succinate production is obligatory linked to the exponential growth phase of the microorganism by genetically altering the metabolic pathways of the cell. The genetic alterations make succinate an obligatory product during the growth phase. Sets of metabolic alterations or transformations that result in elevated levels of succinate biosynthesis during exponential growth are exemplified in Table 1. Each alteration within a set corresponds to the requisite metabolic reaction that should be functionally disrupted. Functional disruption of all reactions within each set results in the obligatory production of succinate by the engineered strain during the growth phase. The corresponding reactions to the referenced alterations in Table 1, and the gene or genes that potentially encode them in E. coli, are set forth in Table 2. Table 3 provides the full biochemical names for the reactants, cofactors and products referenced in the reactions of Table 2.

For example, for each strain exemplified in Table 1, the metabolic alterations that can be generated for growth coupled succinate production are shown in each row. These alterations include the functional disruption of from one to six or more reactions. In particular, 187 strains are exemplified in Table I that have non-naturally occurring metabolic genotypes. Each of these non-naturally occurring alterations result in an enhanced level of succinate production during the exponential growth phase of the microorganism compared to a wild-type strain, under appropriate culture conditions. Appropriate conditions include, for example, those exemplified further below in the Examples such as particular carbon sources or reactant availabilities and/or adaptive evolution.

Given the teachings and guidance provided herein, those skilled in the art will understand that to disrupt an enzymatic reaction it is necessary to disrupt the catalytic activity of the one or more enzymes involved in the reaction. Disruption can occur by a variety of means including, for example, deletion of an encoding gene or incorporation of a genetic alteration in one or more of the encoding gene sequences. The encoding genes targeted for disruption can be one, some, or all of the genes encoding enzymes involved in the catalytic activity. For example, where a single enzyme is involved in a targeted catalytic activity disruption can occur by a genetic alteration that reduces or destroys the catalytic activity of the encoded gene product. Similarly, where the single enzyme is multimeric, including heteromeric, disruption can occur by a genetic alteration that reduces or destroys the function of one or all subunits of the encoded gene products. Destruction of activity can be accomplished by loss of the binding activity of one or more subunits in order to form an active complex, by destruction of the catalytic subunit of the multimeric complex or by both. Other functions of multimeric protein association and activity also can be targeted in order to disrupt a metabolic reaction of the invention. Such other functions are well known to those skilled in the art. Further, some or all of the functions of a single polypeptide or multimeric complex can be disrupted according to the invention in order to reduce or abolish the catalytic activity of one or more enzymes involved in a reaction or metabolic modification of the invention. Similarly, some or all of enzymes involved in a reaction or metabolic modification of the invention can be disrupted so long as the targeted reaction is destroyed.

Given the teachings and guidance provided herein, those skilled in the art also will understand that an enzymatic reaction can be disrupted by reducing or eliminating reactions encoded by a common gene and/or by one or more orthologs of that gene exhibiting similar or substantially the same activity. Reduction of both the common gene and all orthologs can lead to complete abolishment of any catalytic activity of a targeted reaction. However, disruption of either the common gene or one or more orthologs can lead to a reduction in the catalytic activity of the targeted reaction sufficient to promote coupling of growth to succinate biosynthesis. Exemplified herein are both the common genes encoding catalytic activities for a variety of metabolic modifications as well as their orthologs. Those skilled in the art will understand that disruption of some or all of the genes encoding a enzyme of a targeted metabolic reaction can be practiced in the methods of the invention and incorporated into the non-naturally occurring microorganisms of the invention in order to achieve the growth-coupled succinate production.

Therefore, the invention further provides a non-naturally occurring microorganism having a set of metabolic modifications obligatory coupling succinate production to growth of said microorganism. The set of metabolic modifications include disruption of one or more genes encoding an enzyme catalyzing each reaction selected from the set of reactions comprising:

(a) ADHEr (adhE), LDH_D (ldhA)

(b) ADHEr (adhE), LDH_D (ldhA), PTAr (ackA-pta)

(c) PFL (pfl), LDH-D (ldhA)

(d) PFL (pfl) LDH_D (ldhA), ADHEr (adhE)

(e) PTAr (ackA-pta), PYK (pykA, pykF), ATPS4r (atp), SUCD1i (sdh)

(f) PTAr (ackA-pta), PYK (pyka, pykF), GLCpts (ptsG), or

(g) PTAr (ackA-pta), PYK (pykA, pykF), GLCpts (ptsG), ADHEr (adhE), LDH_D (ldhA),

wherein said microorganism exhibits stable growth-coupled production of succinate. The common names for the genes encoding the enzymes responsible for catalyzing the specified reactions are shown in parenthesis. The non-naturally occurring microorganisms having the metabolic modification (e) PTAr, PYK, ATPS4r, SUCD1i, (f) PTAr, PYK, GLCpts, or (g) PTAr, PYK, GLCpts, ADHEr, LDH_D can further include disruption of at least one gene encoding an enzyme catalyzing the reaction DHAPT (dha). Note that pykA and pykF are genes encoding separate enzymes potentially capable of carrying out the PYK reaction. Thus at least one and possibly both pykA and pykF must be removed to prevent PYK from uncoupling succinate production from cell growth. Alternatively, the reactions PFL, ATPS4r, SUCD1i, and DHAPT are carried out by protein complexes encoded by multiple genes. Deleting one or a combination of genes from the pfl, atp, sdh, or dha gene clusters, respectively, are thus sufficient for disrupting the ATPS4r, SUCD1i, or DHAPT reactions.

Briefly, with respect to the genes exemplified above and their relationship to their cognate subunits within multimeric complexes, their orthologs and the reactions catalyzed by their gene products, ADHr is catalyzed by the enzyme encoded by one gene, b 1241 (adhE). LDH_D is encoded by the product of one gene, b1380 (ldhA), which has an ortholog b2133. PFL activity requires enzyme subunits encoded by four genes, b0902, 0903, b3952, and b3951 (represented collectively as pfl). b3114 is an ortholog to b0903. PTAr is encoded by the product of one gene, b2297(ackA-pta), which has an ortholog b2458. PYK is encoded by the product of two different orthologous genes: b1854 (pykA) and b1676 (pykF). Both of these have been shown to be active in E. coli. SUCD1i activity requires enzyme subunits encoded by four genes, b0721, b0722, b0723, and b0724 (represented collectively as sdh). ATPS4r is catalyzed by a multisubunit enzyme encoded by the nine genes b373 I -b3739, which are represented collectively as atp. DHAPT activity requires enzyme subunits encoded by the five genes b1198, b1199, b1200, b2415, and b2416 (represented collectively as dha). GLCpts activity requires enzyme subunits encoded by nine genes: b2415, b2416, b2417, b1817, b1818, b1819, b1101, b0679, and b1621 (represented collectively as ptsG). Since the reactions ATPS4r, SUCD1i, PFL, and DHAPT are carried out by protein complexes encoded by multiple genes, deleting one or a combination of genes from the atp, sdh, pfl, or dha gene clusters, respectively, are thus sufficient for disrupting the reactions. In the remaining cases, the gene responsible for the primary reaction activity in E. coli was chosen, based on information in the literature.

Accordingly, the invention also provides a non-naturally occurring microorganism having a set of metabolic modifications obligatory coupling succinate production to growth of the microorganism, the set of metabolic modifications comprising disruption of one or more genes selected from the set of genes comprising: (a) adhE, ldhA; (b) adhE, ldhA, acka-pta; (c) pfl, ldhA; (d)pfl, ldhA, sdhA; (e) acka-pta, pykF, atpF, sdhA; (f) acka-pta, pykF, ptsG, or (g) ackA-pta, pykF, ptsG, adhE, ldhA, or an ortholog thereof, wherein the microorganism exhibits stable growth-coupled production of succinate. Additionally provided is a non-naturally occurring microorganism having the genes encoding the metabolic modification (e) ackA-pta, pykF, atpF, sdhA that further includes disruption of at least one gene selected from pykA, atpH, sdhB or dhaKLM; a non-naturally occurring microorganism having the genes encoding the metabolic modification (f) ackA-pta, pykF, ptsG that further includes disruption of at least one gene selected from pykA or dhaKLM, or a non-naturally occurring microorganism having the genes encoding the metabolic modification (g) ackA-pta, pykF, ptsG, adhE, ldhA that further includes disruption of at least one gene selected from pykA or dhaKLM.

The non-naturally occurring microorganisms of the invention can be employed in the growth-coupled production of succinate. Essentially any quantity, including commercial quantities, can be synthesized using the growth-coupled succinate producers of the invention. Because the the microorganisms of the invention obligatory couple succinate to growth continuous or near-continuous growth processes are particularly useful for biosynthetic production of succinate. Such continunous and/or near continuous growth processes are described above and exemplified below in the Examples. Continuous and/or near-continuous microorganism growth process also are are well known in the art. Briefly, continuous and/or near-continuous growth process involve maintaining the microorganism in an exponential growth or logarythimic phase. Procedures include using apparatuses such as the Evolugator™ evolution machine (Evolugate LLC, Gainesville, Fla.), fermentors and the like. Additionally, shake flask fermentation and grown under microaerobic conditions also can be employed. Given the teachings and guidance provided herein those skilled in the art will understand that the growth-coupled succinate producing microorganisms can be employed in a variety of different settings under a variety of different conditions using a variety of different processes and/or apparatuses well known in the art.

Generally, the continuous and/or near-continuous production of succinate will include culturing a non-naturaly occurring growth-coupled succinate producing organism of the invention in sufficient neutrients and medium to sustain and/or nearly sustain growth in an exponentially phase. Continuous culture under such conditions can be include, for example, a day, 2, 3, 4, 5, 6 or 7 days or more. Additionally, continuous culture can include I week, 2, 3, 4 or 5 or more weeks and up to several months. In is to be understood that the continuous and/or near-continuous culture conditions also can include all time intervals in between these exemplary periods.

Succinate can be harvested or isolated at any time point during the continuous and/or near-continuous culture period exemplified above. As exemplified below in the Examples, the longer the microorganisms are maintained in a continuous and/or near-continuous growth phase, the proportionally greater amount of succinate can be produced.

Therefore, the invention provides a method of producing succinate coupled to the growth of a microorganism. The method includes: (a) culturing under exponential growth phase in a sufficient amount of neutrients and media a non-naturally occurring microorganism comprising a set of metabolic modifications obligatory coupling succinate production to growth of the microorganism, the set of metabolic modifications comprising disruption of one or more genes selected from the set of genes comprising:

(1) adhE, ldhA

(2) adhE, IdhA, acka-pta

(3) pfl, idhA

(4) pfl, idhA, adhE

(5) ackA-pta, pykF, atpF, sdhA

(6) ackA-pta, pykF, ptsG, or

(7) ackA-pta, pykf, ptsG, adhE, ldhA,

or an ortholog thereof, wherein the microorganism exhibits stable growth-coupled production of succinate, and (b) isolating succinate produced from the non-naturally occurring microorganism. The genes encoding the metabolic modification (5) ackA-pta, pykF, atpF, sdhA can further comprise disruption of at least one gene selected from pyka, alpH, sdhB or dhaKLM. The genes encoding the metabolic modification (6) ackA-pta, pykf, ptsG can further comprise disruption of at least one gene selected from pyka or dhaKLM. The genes encoding the metabolic modification (7) acka-pta, pykF, ptsG, adhE, ldhA further comprises disruption of at least one gene selected from pykA or dhaKLM.

It is understood that modifications which do not substantially affect the activity of the various embodiments of this invention are also included within the definition of the invention provided herein. Accordingly, the following examples are intended to illustrate but not limit the present invention.

### EXAMPLE I

Microorganisms Having Growth-coupled Production of Succinate

In this Example, the metabolic engineering strategies identified by the methods described previously are described. Overall, more than one hundred strategies were identified. A detailed listing of the identified metabolic alterations leading to growth-coupled succinate production can be found in Tables 1-3 below (following the Examples), which describe the reaction deletion combinations identified by OptKnock (Table 1), the stoichiometry and genes corresponding to each reaction mentioned in Table 1 (Table 2), and the list of metabolite names corresponding to their abbreviations in Table 2 (Table 3). Particularly useful designs for the purpose of demonstrating the methods described herein were placed into three categories: (1) intuitive designs (low risk), (2) non-intuitive aerobic design (medium risk), (3) non-intuitive anaerobic design (higher risk). Depending on the strategy, either aerobic or anaerobic conditions were examined for a basis glucose uptake of 10 mmol/gDW/hr. The solution boundaries for each design are obtained by separately maximizing and minimizing succinate production at every feasible growth rate. The rightmost portion of the solution boundary corresponds to the “optimal growth” solution which is synonymous with the maximum biomass yield. Because of the linearity of the system, if the glucose uptake rate were to be different than the arbitrary basis, the results would scale proportionally. Procedures for the construction and culturing of strains identified as having growth-coupled succinate production also are described further below.

Intuitive Designs

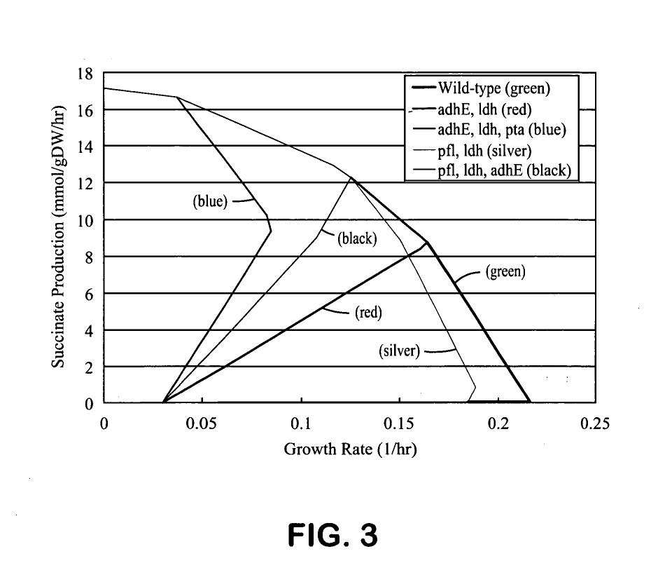

The biochemical production limits for four of the intuitive strain designs identified by the OptKnock technology are described in FIG. 3. The boundaries assume anaerobic conditions in the presence of ample carbon dioxide. The risk associated with evaluating these strains was deemed minimal as three of them (i.e., all but the strain termed pfl, ldh, adhE) have been constructed by others. However, none of the three specifically constructed strains were shown to have growth growth-coupled succinate production nor were they shown to have stable growth-coupled production of succinate. Moreover, none of the three specifically constructed strains were subjected to adaptive evolution as described herein to further augment growth-coupled succinate production. Therefore, the strains of the invention adhE, ldh; adhE, ldh, pta, and pfi, ldh are distinct from those previously constructed strains.

A brief description of the suggested designs will follow. The exchange fluxes corresponding to the maximum growth simulation of each suggested mutant are shown in Table 4.

adhE, ldhA (blue)-This particular mutant was mentioned in Sanchez et al., Metab Eng, 7: 229-39 (2005) and Sanchez et al., Biotechnol Prog, 21: 358-65 (2005), and is called SBS 11 OMG. A heterologous pyc was expressed in the final production strains. The authors reported a succinate yield of only 0.2 mol/mol for the double mutant with the control plasmid without pyc. With the pyc plasmid, the yield increased to 1.3 mol/mol. A yield of 0.9 mol/mol is expected post-evolution. adhE, ldhA, pta (red)-These deletions are present in the best anaerobic production strain described in Sanchez et al., Metab Eng, 7: 229-39 (2005). The actual production strain also harbors the pyc plasmid and contains another knockout, iclR, a regulatory gene that represses the glyoxylate shunt. Simulations reveal the glyoxylate shunt to be of minimal importance. The overexpression of pyc is the most likely reason for the high published yield of 1.6 mol/mol. Based on in silico analysis, the yield of the triple deletion mutant is expected to be 0.9 mol/mol after adaptive evolution, a significant decrease from the 1.6 mol/mol observed previously, Sanchez et al., Metab Eng, 7: 229-39 (2005).

pfl, ldhA (gray)-A pfl, ldhA strain was mentioned in Donnelly et al., Appl Biochem Biotechnol, 70-72: 187-98 (1998) and the original OptKnock paper, Burgard et al., Biotechnol Bioeng, 84: 647-57 (2003). The strain, called NZN111, was unable to ferment glucose but eventually had a spontaneous mutation in ptsG that allowed it to ferment glucose. Hong and Lee, Hong et al., Biotechnol Bioeng, 74: 89-95 (2001), overexpressed malic enzyme in NZN111 and achieved 30-40% yields of succinate. An evolution experiment with NZN111 or any other pfl, ldh mutant strain is particularly useful because succinate production at the endpoint should be small.

pfl, ldhA, adhE (green)-This mutant was suggested in the original OptKnock paper, Burgard et al., Biotechnol Bioeng, 84: 647-57 (2003). This strain should have ˜1.2 mol/mol glucose yield at the evolution endpoint with succinate and acetate being the major products. This strain would be interesting because it should have the highest succinate yield at the evolution endpoint. The pfl deletion is counterintuitive because the production of formate is widely considered a source of reducing equivalents and thus beneficial for succinate formation. The stoichiometric model disputes this because formate dehydrogenase does not operate anaerobically in simulations. Deleting fdhF (formate dehydrogenase) from the pyc containing SBS 110MG (ldhA, adhE mutant) decreased the succinate yield from 1.2 to 0.9 mol/mol Sanchez et al., Biotechnol Prog, 21:358-65 (2005).

TABLE 4
Exchange fluxes for the optimal growth simulations assuming anaerobic
conditions and a basis glucose uptake rate of 10 mmol/(gDW · hr).
Positive values indicate production, negative values indicate consumption.
Molar yields of all chemicals can be obtained by dividing the entry
by the basis glucose uptake rate (i.e., 10).

| | adhE, ldhA | adhE, ldhA, pta | pfl, ldhA | pfl, ldhA, adhE |
|---|---|---|---|---|
| | blue | red | gray | green |
| | 1/hr | 1/hr | 1/hr | 1/hr |
| Biomass | 0.16 | 0.08 | 0.19 | 0.13 |
| | mmol/ | mmol/ | mmol/ | mmol/ |
| | (gDW · hr) | (gDW · hr) | (gDW · hr) | (gDW · hr) |
| Glucuse | −10.00 | −10.00 | −10.00 | −10.00 |
| CO2 | −8.77 | −9.37 | 16.25 | −5.88 |
| Ammonia | −1.75 | −0.90 | −2.01 | −1.34 |
| Phosphate | −0.15 | −0.08 | −0.17 | −0.11 |
| Sulfate | −0.04 | −0.02 | −0.04 | −0.03 |
| Acetate | 8.78 | 0.05 | 0.00 | 5.83 |
| Ethanol | 0.00 | 0.00 | 16.29 | 0.00 |
| Formate | 9.44 | 0.41 | 0.00 | 0.01 |
| Glyclt | 0.01 | 0.00 | 0.01 | 0.01 |
| Pyruvate | 0.00 | 9.29 | 0.00 | 0.00 |
| Succinate | 8.74 | 9.37 | 0.84 | 12.25 |
| H+ | 37.21 | 29.27 | 3.41 | 31.49 |
| H2O | 3.44 | 11.08 | 4.78 | 9.03 |

Non-intuitive Aerobic Design

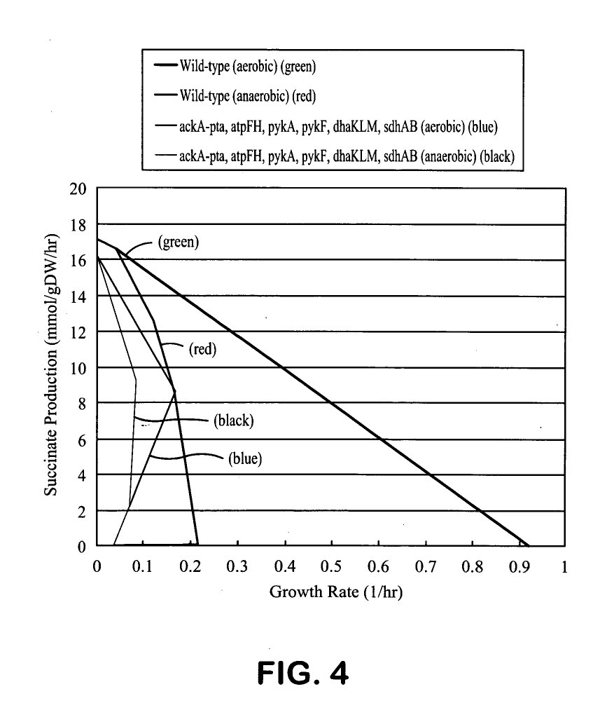

This strain design requires six deletions (i.e., acka-pta, pyka, pykF, atpFH, sdhAB, dhaKLM) for growth-coupled succinate formation under aerobic conditions. The reactions disabled by the deletions include PTAr, PYK, ATPS4r, SUCD1i, and DHAPT. The biochemical production limits for the strain design are shown in FIG. 4 and optimal growth simulation results are provided in Table 5. Note that the deletions cause a reduction in the maximum theoretical succinate yield. Nevertheless, this design is particularly useful because it allows the deletion of atpFH for aerobic biochemical production to be further characterized.

The rational behind the dhaKLM deletion is that the reactions F6PA (f6p ←→dha+g3p) and DHAPT (dha+pep→dhap+pyr) take on large fluxes when included in the simulations. These reactions provide a means of converting pep to pyruvate, which can be undesirable for succinate formation. This pathway seems realistic from an energetic standpoint because pep is a very high-energy compound. In addition, the enzymes responsible for both reactions have been characterized, Schurrnann et al., J Biol Chem, 276: 11055-61 (2001); Gutknecht et al., Embo J, 20: 2480-6 (2001). Although these enzymes could likely not carry full glycolytic fluxes, the most conservative approach is to ensure these fluxes are non-operative by deleting dhaKLM. In practice, this deletion may or may not be necessary. Simulations also revealed that ptsG could be removed as an alternative to dlhaKLM, but a strain harboring both the ptsG and atpFH deletions may encounter growth difficulties due to its complete reliance on hexokinase for glucose uptake.

TABLE 5
Exchange fluxes for the optimal growth simulations assuming a
basis glucose uptake rate of 10 mmol/(gDW · hr). Positive
values indicate production, negative values indicate consumption.
Molar yields of all chemicals can be obtained by dividing the
entry by the basis glucose uptake rate (i.e., 10).

| | aerobic | anaerobic |
|---|---|---|
| | red | green |
| | 1/hr | 1/hr |
| Biomass | 0.17 | 0.08 |
| | mmol/(gDW · hr) | mmol/(gDW · hr) |
| Glucuse | −10.00 | −10.00 |
| CO2 | 8.62 | −1.07 |
| Ammonia | −1.80 | −0.90 |
| Oxygen | −17.18 | 0.00 |
| Phosphate | −0.15 | −0.08 |
| Sulfate | −0.04 | −0.02 |
| Acetate | 0.10 | 0.05 |
| Ethanol | 0.18 | 8.30 |
| Formate | 9.61 | 11.82 |
| Pyruvate | 0.00 | 0.00 |
| Succinate | 8.55 | 7.26 |
| H+ | 28.36 | 27.17 |
| H20 | 20.57 | −0.32 |

Non-intuitive Anaerobic Design

This strain design requires five deletions (i.e., ackA-pta, pykA, pykF, ptsG, dhaKLM) for growth-coupled succinate formation under anaerobic conditions. The reactions disabled by the deletions include PTAr, PYK, GLCpts, and DHAPT. Again, the DHAPT deletion (i.e., dhaKLM) can be optional in practice. The deletion set is expected to result in significant metabolic changes such as the forced reliance on atypical sources of pyruvate (e.g., entner doudoroff pathway, serine deaminase, malic enzyme, etc.). This design exhibits some drawbacks becaue ptsG, pyka, pykF mutants can be unviable when grown on glucose minimal media, Ponce et al., J Bacteriol, 177: 5719-22 (1995). Simulations contradict this finding and indicate that high biomass yields are possible for aerobic growth on glucose. This result indicates that an evolutionary strategy can be employed to enhance the strain adjust to the deletions if it encounters the growth difficulties anticipated for a pykA, pykF, ptsG mutant.

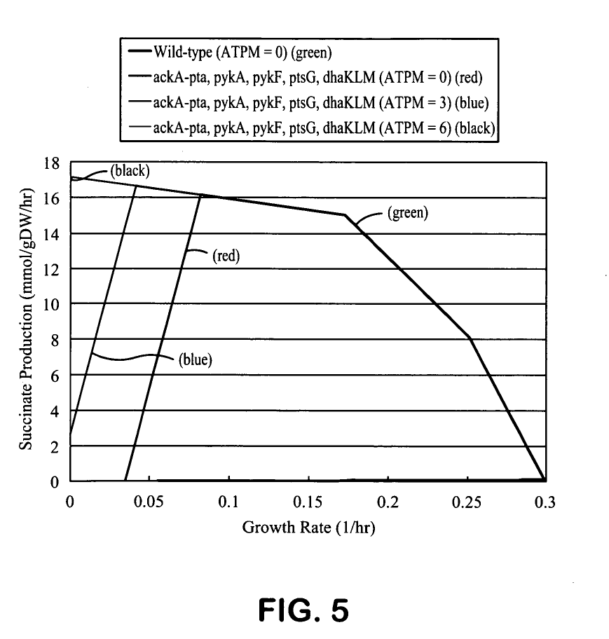

Anaerobically, simulations reveal this strain to be deficient in meeting the standard 7.6 mmol/gDW/hr non-growth associated ATP maintenance requirement if the glucose uptake rate is assumed 10 mmol/gDW/hr. The effect of relaxing the ATP maintenance requirement on the calculated solution boundaries is examined in FIG. 5. Here it is shown that if this penta-mutant could be grown anaerobically (i.e., applying growth selection pressures to force the cells to lessen their maintenance energy requirements), extremely high yields of succinate must accompany optimal growth. Thus this design has a huge potential upside that outweighs its risk. In addition, the introduction of a futile cycle can be harnessed to drive the performance of this strain to the near theoretical succinate yields denoted by the leftmost upper portion of the solution boundaries depicted in FIG. 5. The exchange fluxes of the maximum growth simulation assuming no energy maintenance requirement and anaerobic conditions are shown in Table 6.

TABLE 6
Exchange fluxes for the optimal anaerobic growth simulations assuming
a basis glucose uptake rate of 10 mmol/(gDW · hr) and a non-growth
associated maintenance requirement of zero. Positive values indicate
production, negative values indicate consumption. Molar yields
of all chemicals can be obtained by dividing the entry by the basis
glucose uptake rate (i.e., 10).

| | 5 deletions |
|---|---|
| | blue |
| | 1/hr |
| Biomass | 0.08 |
| | mmol/(gDW · hr) |
| Glucuse | −10.00 |
| CO2 | −7.89 |
| Ammonia | −0.87 |
| Oxygen | 0.00 |
| Phosphate | −0.07 |
| Sulfate | −0.02 |
| Acetate | 0.05 |
| Ethanol | 0.00 |
| Formate | 0.00 |
| Glyclt | 0.004 |
| Pyruvate | 0.00 |
| Succinate | 16.10 |
| H+ | 33.00 |
| H2O | 9.94 |

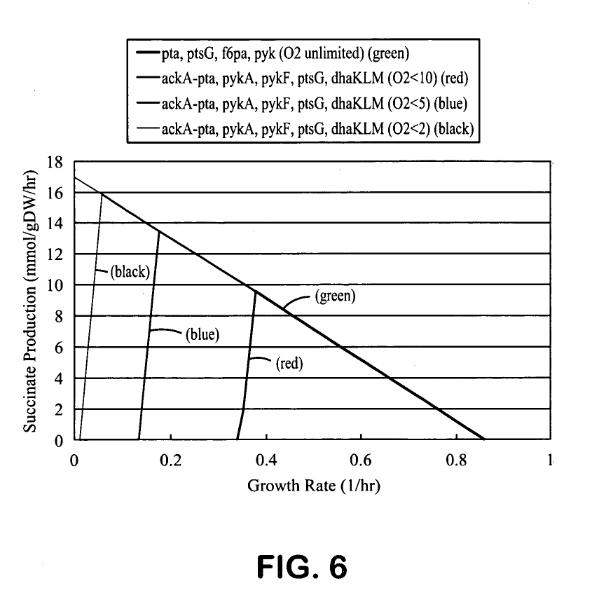

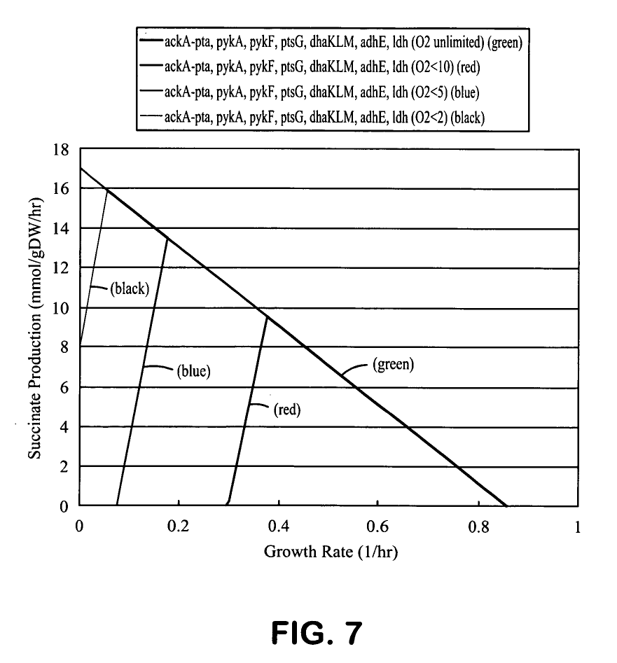

In the specific instance where the newly constructed stain is viable in an aerobic environment but not sufficiently viable under anaerobic conditions, an evolutionary strategy involving decreasing aeration rates can be applied. The effect of gradually decreasing the rate of oxygen uptake on the succinate production limits is depicted in FIG. 6. Note that decreasing aeration leads to an optimal growth point increasingly closer to the maximum theoretical succinate yield (top left). A tighter coupling of succinate formation to cell growth can be achieved with the additional deletion of adhE and ldh as shown in FIG. 7. These genes catalyze the ADHEr and LDH_D reactions, respectively. Also, the indicated viability of the strain with or without the adhE, dhaKLM, ldh deletions under both anaerobic and aerobic conditions further indicates that a dual-phase fermentation strategy involving an aerobic growth phase (black line) followed by an oxygen-limited production phase (colored lines) can be employed.

Production and Validation of Growth-Coupled Succinate Strains

In order to confirm the computational predictions of the methods of the invention, the strains are constructed, evolved, and tested. A growth conditioning phase of between one and six weeks, generally about three weeks is allotted for more complicated designs. Simpler designs can be conditioned for corresponding shorter periods of time.

Strain Construction: Escherichia coli K-12 MG1655 is used as one reference wild-type strain into which the deletions are introduced. The knockouts are integrated, for example, one-by-one into the recipient strain allowing the accumulation of several deletions. The deletion methodology completely removes the gene targeted for removal so as to avoid the possibility of the constructed mutants reverting back to their wild-type. In one study, two of the four predicted non-intuitive designs are constructed and assayed for growth-coupled production of succinate. Here two strains can be constructed requiring at most three deletions: (1) pfl, ldhA and (2)pfl, ldhA, adhE. For other studies, four common knockouts are constructed: ackA-pta, pyk,A, pykF, dhaKLM. In one further study, the additional deletion of atpFH and sdhAB is performed, while in an alternative study additional deletion of ptsG is performed. At most seven deletions are incorporated for confirmatory testing of the non-intuitive designs. Overall, at most ten deletions can be performed for the construction all desired non-intuitive strains and studies.

Adaptive Evolution: The adaptive evolution procedure involves maintaining the cells in prolonged exponential growth by the serial passage of batch cultures into fresh medium before the stationary phase is attained. Briefly, the cells are allowed to reach mid-exponential growth (A₆₀₀=0.5) before being diluted and passed to fresh medium (i.e., M9 minimal media with 2 g/L carbon source). This process is repeated, allowing for about 500 generations for each culture. Culture samples are taken, frozen with liquid nitrogen, and the optical culture density recorded for each day throughout the course of the evolutions. The conditions required for each evolution are summarized on table 7. The evolutions are performed in triplicate (i.e., 18 evolutions total) due to differences in the evolutionary patterns witnessed previously Donnelly et al., Appl Biochem Biotechnol 70-72: 187-98 (1998); Vemuri et al., Appl Environ Microbiol 68:1715-27 (2002), that could potentially result in one strain having superior production qualities over the others. The adaptive evolution step can take up to about two months or more. The adaptive evolution step also can be less than two months depending on the strain design, for example.

TABLE 7
Desired conditions for each evolution. The evolutions
will be carried out in triplicate.

| | | | |
|---|---|---|---|
| Wild-type (aerobic) | Glucose | Aerobic | CO2 optional |
| | Minimal | | |
| Wild-type (anaerobic) | Glucose | Anaerobic | CO2 required |
| | Minimal | | |
| Intuitive Anaerobic Design #1 | Glucose | Anaerobic | CO2 required |
| (pfl, ldh) | Minimal | | |
| Intuitive Anaerobic Design #2 | Glucose | Anaerobic | CO2 required |
| (pfl, ldh, adhE) | Minimal | | |
| Nonintuitive Aerobic Design | Glucose | Aerobic | CO2 optional |
| (ackA-pta, dhaKLM, pykF, | Minimal | | |
| pykA, sdhAB, atpFH) | | | |
| Nonintuitive Anaerobic Design | Glucose | Anaerobic | CO2 required |
| (ackA-pta, dhaKLM, pykF, | Minimal | | |
| pykA, ptsG) | | | |

Validation: The growth rate (GR), substrate uptake rate (SUR), and oxygen uptake rate (OUR) (if aerobic) is sampled every ten days throughout the course of the evolutions. Pre-cultures are grown overnight and used as innoculum for a fresh batch culture for which measurements are taken during exponential growth. The GR is determined by measuring optical density using a spectrophotometer (A₆₀₀ and A₄₂₀), the SUR by monitoring carbon source depletion over time by HPLC, and the OUR by measuring the dissolved oxygen depletion in a respirometer using a polarographic dissolved oxygen probe. Succinate and byproduct production are quantified by HPLC or an enzymatic assay. Measurements are taken for each evolved strain in triplicate at ten-day intervals. The testing can run concurrently with the evolutions.

Evaluation of Strain Stability: The growth-coupled biochemical production concept behind the OptKnock approach results in the generation of genetically stable overproducers. The strain with the best succinate production characteristics and at least satisfactory correspondence to the model predictions are grown in a chemostat for one month to confirm its long-term stability. The chemostat cultivation is performed using M9 minimal media in a 1.3-L benchtop fermentor (New Brunswick Scientific, Edison, N.J.) at a working volume of approximately 600 mL. Carbon source concentrations are adjusted to achieve a cell density corresponding to A₆₀₀ =1.0, and the dilution rate will be set to approximately 80% of the maximum growth rate to avoid any possibility of washout. Sterile air is used for aerobic growth, and the dissolved oxygen is maintained at >95% of saturation using the agitation rate. If OptKnock suggests an anaerobic growth environment, the reactor will be continuously sparged with a nitrogen/CO₂ mixture to ensure that DO levels remain below detection levels. CO₂ also is provided in the medium in the form of sodium bicarbonate (NaHCO₃). The metabolic behavior is assayed each week as described above.

Conclusions

Described herein is the application of the OptKnock methodology for generating useful gene deletion targets. Multiple deletion strategies were enumerated for establishing the coupling between succinate production and E. coli growth. This methodology is applicable to a wide variety of cells and microorganisms other than E. coli and also can utilize metabolic modeling and simulation systems other than OptKnock. Construction and validation of two relatively intuitive anaerobic designs (3 and 4 below), one non-intuitive aerobic design (5), and one non-intuitive anaerobic design (6) also is described. The procedures also include a detailed evaluation of the capabilities of adaptive evolution to corroborate enhance the production characteristics of each biocatalyst. Specifically, seven strain designs of those strains identified as having growth-coupled production of succinate were selected whose implementation can lead to the coupling of succinate biosynthesis to growth:

Deletion strategies selected for experimental validation:

1) adhE, ldhA

2) adhE, ldhA, acka-pta

3) pfl, ldhA

4) pfi, ldhA, adhE

5) acka-pta, pykA, pykF, atpFH, sdhAB, dhaKLM

6) acka-pta, pyka, pykF, ptsG, dhaKLM

7) acka-pta, pyka, pykF, ptsG, dhaKLM, adhE, ldhA

The analysis described herein also indicates that an evolutionary approach can improve the succinate production characteristics of all suggested strains. The results of the analysis described herein also indicate that several of the specific strains described above can still exhibit growth-coupled succinate production when less than the complete set of listed metabolic modifications are incorporated. Thus, the following list specifies one minimal set of deletions required for each of the designs:

Minimal set of deletions required for the above designs:

1) adhE, ldhA

2) adhE, ldhA, ackA-pta

3) pfi, ldhA

4) pfl, ldhA, adhE

5) ackA-pta, pykF, atp, sdh

6) ackA-pta, pykF, ptsG

7) ackA-pta, pykF, ptsG, adhE, ldhA where sdh and atp represents any deletion capable of eliminating succinate dehydrogenase (SUCD1i) and ATP synthase (ATPS4r) activity, respectively, from E. coli. The combined computational and engineering platform described herein is generally applicable to any stoichiometric model organism and the teachings and guidance provided herein will allow those skilled in the art to design and implement sets of genetic manipulations for metabolic alterations that lead to the growth-coupled production of any biochemical product.

### EXAMPLE II

Microorganisms Having Growth-coupled Production of Succinate

This Example describes the construction and performance of two in silico designed strains described in Example I for the growth-coupled production of succinate.

Described below are the methods used to construct and characterize organisms containing two of the previously described strain designs. Briefly, one strain, termed AB3, included deletions in adhE, pflA, and ldhA, while the second strain, termed AB4, included deletions in ackA-pta, dhaKLM, ptsG, pykA, and pykF, both described previously.

Strain Characterization Pre-Evolution

The first design involved the simultaneous removal of pfl, ldh, and adhE. The theoretical production limits for the proposed pfl, ldh, adhE triple mutant, obtained by separately maximizing and minimizing the succinate yield at every feasible growth rate, are compared to those of the wild-type strain in FIG. 3. The regions encompassed by the green and black lines denote succinate and biomass production rates theoretically achievable to E. coli. The rightmost portion of the production limits corresponds to the “optimal growth” solution, which is synonymous with the maximum biomass yield. Because of the linearity of the system, if the glucose uptake rate were to be different than the arbitrary basis, the results would scale proportionally. As described previously in Example 1, completely anaerobic fermentation is predicted to be feasible for apfl, ldh, adhE triple mutant and lead to a succinate molar yield of 1.2 mol/mol at optimal growth. Note also that succinate production is predicted to be tightly coupled to cell growth in this strain with a non-zero succinate yield becoming essential at a growth rate above 25% of the in silico predicted maximum. Lastly, the maximum theoretical succinate yield is not affected by the knockouts and thus this design strategy will not negatively impact further metabolic engineering of this strain.

The second OptKnock-derived design requires five deletions (i.e., acka-pta, pyka, pykF, ptsG, dhaKLM) for growth-coupled succinate formation under anaerobic conditions. As described previously in Example I, the deletion set is expected to result in significant metabolic changes such as the forced reliance on atypical sources of pyruvate (e.g., entner doudoroff pathway, serine deaminase, malic enzyme, etc.).

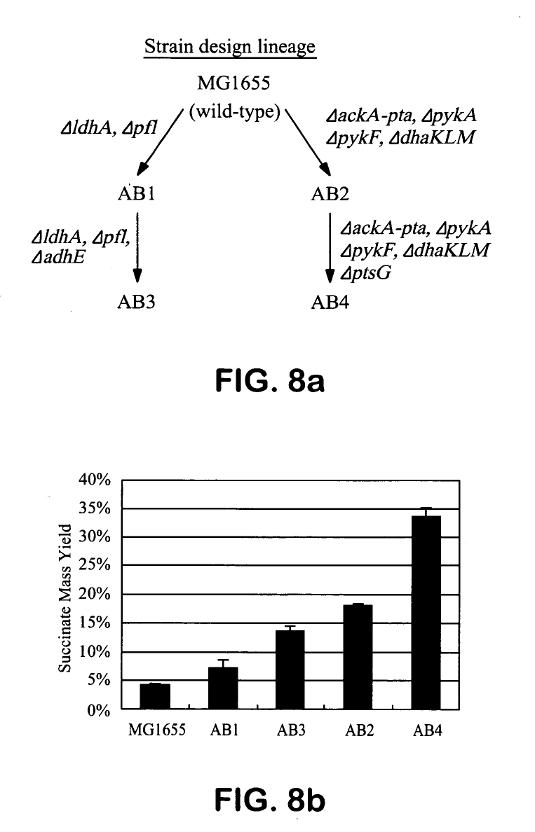

Briefly, strains were constructed using Escherichia coli K-12 MG1655 as the wild-type strain into which the deletions were introduced. The knockouts were integrated one-by-one into the recipient strain allowing the accumulation of several deletions. The strains were constructed by incorporating in-frame deletions using homologous recombination via the X Red recombinase system of Datsenko and Wanner, Datsenko et al., Proc. Nat. Acad. Sci. USA., 6640-6645 (2000). No drug resistance markers remain after each deletion, allowing multiple mutations to be accumulated in the target strains. In addition, complete removal of the targeted gene avoids the possibility of the constructed mutants reverting back to their wild-type. The first strain was constructed with deletions in pfl, ldh, and adhE while the second strain had deletions in ackA-pta, pykA, pykF, dhaKLM, and ptsG. The strategy used to construct these strains is outlined in FIG. 8a.

Strain performance was quantified by performing shake flask fermentations with all strains both before and after the evolutions. Anaerobic conditions were obtained by first sparging the medium with nitrogen and then sealing the flasks with a septum and crimp-cap. For strains where growth was not observed anaerobically, microaerobic conditions were applied by poking a small hole through the septum for limited aeration. All experiments were performed using M9 minimal medium at pH 7.0 (6.78 g/L Na₂HPO₄, 3.0 g/L KH₂PO₄, 0.5 g/L NaCl, 1.0 g/L NH₄Cl, 1 mM MgSO₄, 0.1 mM CaCl₂) supplemented with 2 g/L glucose and 20 mM NaHCO₃. Pre-cultures were grown overnight and used as inoculum for a fresh batch culture for which measurements were taken during exponential growth. The growth rate was determined by measuring optical density using a spectrophotometer (600 nm), and the glucose uptake rate by monitoring carbon source depletion over time. Succinate, byproducts, and residual glucose were quantified by HPLC (Shimadzu) with an HPX-087 column (BioRad), using a refractive index detector for glucose and ethanol, and a UV detector for organic acids, Lin et al., Biotechnol. Bioeng., 775-779 (2005). Triplicate cultures were grown for each strain, and all measurements reported are averages of the three.

The performance of the wild-type and engineered strains were characterized prior to the evolution in order to make quantitative conclusions regarding the implementation of the adaptive evolution procedure. The results from this analysis can be found in Table 8. Note that shake flask characterizations were performed for multiple strains constructed in this study, including the intermediates AB1 and AB2 (see FIG. 8a).

TABLE 8
The amounts of glucose consumed, biomass formed, and fermentation products produced,
as well as growth rates for engineered strains. All data are averaged over 3 independent
cultures and the 95% confidence intervals are indicated. N.D., not determined.

| | | MG1655 | AB1 | | AB3 | AB2 | AB4 | |
|---|---|---|---|---|---|---|---|---|
| | | anaerobic | microaerobic | | microaerobic | anaerobic | anaerobic | |
| Glucose | mM | 20.46 ± 0.51 | 36.97 ± 0.68 | | 7.89 ± 0.39 | 11.50 ± 1.69 | 12.12 ± 0.82 | |
| Biomass | g/L | 0.45 ± 0.01 | 0.30 ± 0.00 | | 0.29 ± 0.02 | 0.17 ± 0.02 | 0.20 ± 0.04 | |
| Acetate | mM | 15.48 ± 0.14 | 2.30 ± 0.98 | | 4.37 ± 0.51 | 0.00 ± 0.00 | 1.08 ± 0.21 | |
| Ethanol | mM | 12.26 ± 0.98 | 7.08 ± 1.87 | | 0.00 ± 0.00 | 6.55 ± 0.69 | 0.00 ± 0.00 | |
| Formate | mM | 29.84 ± 0.13 | 0.03 ± 0.04 | | 0.00 ± 0.00 | 8.69 ± 2.05 | 3.57 ± 0.21 | |
| Lactate | mM | 2.20 ± 0.43 | 0.25 ± 0.07 | | 0.13 ± 0.01 | 4.28 ± 0.28 | 2.84 ± 0.22 | |
| Malate | mM | 0.23 ± 0.02 | 0.04 ± 0.07 | | 0.00 ± 0.00 | 0.15 ± 0.06 | 0.00 ± 0.00 | |
| Pyruvate | mM | 0.68 ± 0.07 | 14.08 ± 0.53 | | 11.35 ± 1.08 | 0.10 ± 0.08 | 0.00 ± 0.00 | |
| Succinate | mM | 1.29 ± 0.05 | 4.03 ± 0.81 | | 1.64 ± 0.04 | 3.16 ± 0.45 | 6.21 ± 0.38 | |
| Growth rate | hr-1 | 0.351 ± 0.010 | 0.221 | 0.007 | 0.151 ± 0.005 | N.D. | 0.044 | 0.002 |

The engineered strains exhibited slower growth rates than the wild-type and altered fermentation profiles. AB1 and AB3 were unable to significantly grow under strict anaerobic conditions, so instead were grown microaerobically as described above. This observation is in agreement with literature reports of NZN111, Stols et al., Appl Environ Microbiol, 63: 2695-701 (1997), Hong et al., Biotechnol Bioeng, 74: 89-95 (2001), Stols et al., Appl Biochem Biotechnol, 63-65: 153-8 (1997), which is of the same genotype as AB1. These strains appear to possess a bottleneck at pyruvate as indicated by the relatively high amounts of this metabolic byproduct.

The adhE deletion significantly decreased the amount of glucose consumed in AB3, but also eliminated ethanol production resulting in a higher succinate yield. The shake flask characterizations of strains AB2 and AB4 revealed approximately 4-fold and 10-fold increases in the succinate yield compared to wild-type MG1 655 during completely anaerobic growth (FIG. 8b), although the mutations had a substantial impact on the growth rates and final biomass concentrations.

Adaptive Evolution of Strains

The observed growth of the newly constructed mutants was below what was predicted a priori by the modeling analysis, so the strains were then evolved using the Evolugator™machine. Briefly, adaptive evolution was performed on the wild-type and both engineered strains. The adaptive evolution procedure was carried out by Evolugate, LLC (Gainesville, Fla.), using the Evolugator™ evolution machine. This device maintains the cells in prolonged exponential growth by the serial passage of batch cultures into fresh medium before the stationary phase is attained. By automating optical density measurement and liquid handling, the Evolugator can perform serial transfer at high rates using large culture volumes, thus approaching the efficiency of a chemostat for evolution of cell fitness. However, in contrast to the chemostat which maintains cells in a single vessel, the machine operates by moving from one “reactor” to the next in subdivided regions of a spool of tubing, thus eliminating any selection for wall-growth. Culture samples were taken, frozen with liquid nitrogen, and the optical culture density recorded each day throughout the course of the evolutions.

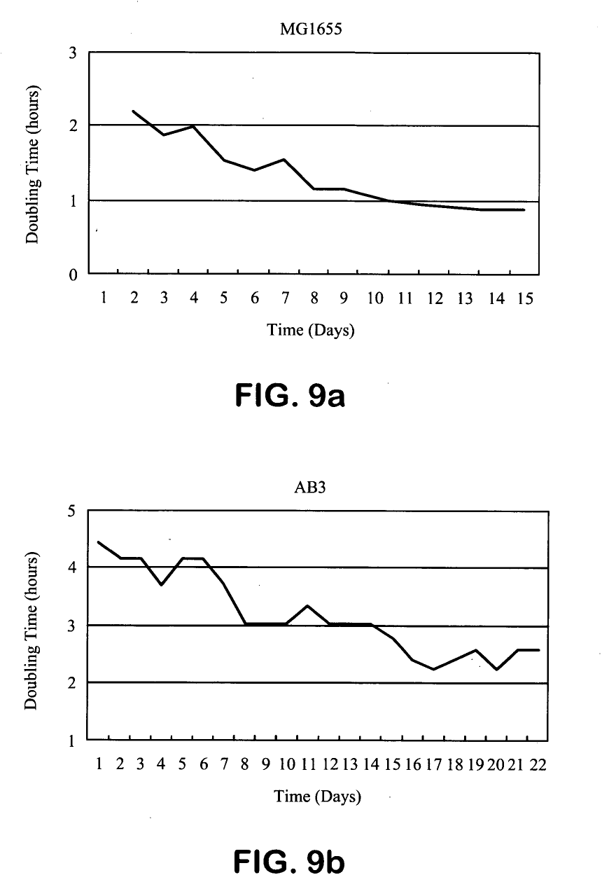

Although anaerobic growth of AB3 was negligible in the laboratory, the ability to grow under this condition was developed by gradually reducing oxygenation during the first few days of the evolution process. As a control experiment to demonstrate that E. coli can be evolved anaerobically, wild-type MG1 655 was run in the Evolugator for a period of 15 days. The approximate doubling time, estimated by the number of dilutions required per day, decreased from 2.2 hr⁻¹ to 0.9 hr⁻¹ as shown in FIG. 9a. To further characterize the growth improvement, triplicate cultures of the original and evolved (EVGO9) strains were grown. The growth rate of the wild-type strain was 0.351±0.002 hr^{-1,} and that of EVG09 was 0.521±0.049 hr^{-1.} Given the satisfactory growth improvements observed for the wild-type strain, strain AB3 was then evolved resulting in similar decreases in doubling times as shown in FIG. 9b. Noticeable decreases in doubling time were observed for both strains.

Further Strain Characterization

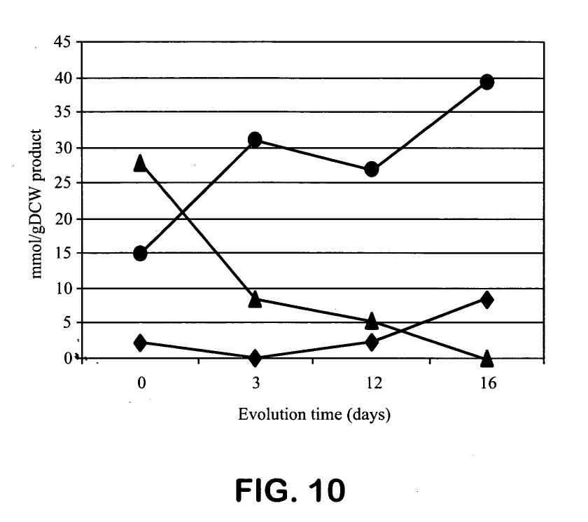

The evolved AB3 was characterized by shake flask fermentations as was done for its un-evolved predecessors described above. Completely anaerobic growth was not significant for this strain indicating that the Evolugator™ growth environment was not completely anaerobic. However, the product profiles of samples taken from the device revealed that significant amounts of fermentation products were produced by strain AB3 indicating that the evolutions were far from fully aerobic and likely microaerobic (FIG. 10). These results indicate that the time spent in the Evolugator™ helped to alleviate the significant pyruvate bottleneck present in the unevolved AB3 strain, as the amount of pyruvate dropped to zero at the end of the run. Pyruvate secretion provides little benefit to the organism because, unlike producing ethanol, lactate, or succinate, no AND is regenerated. In addition, unlike producing acetate, no energy is generated.

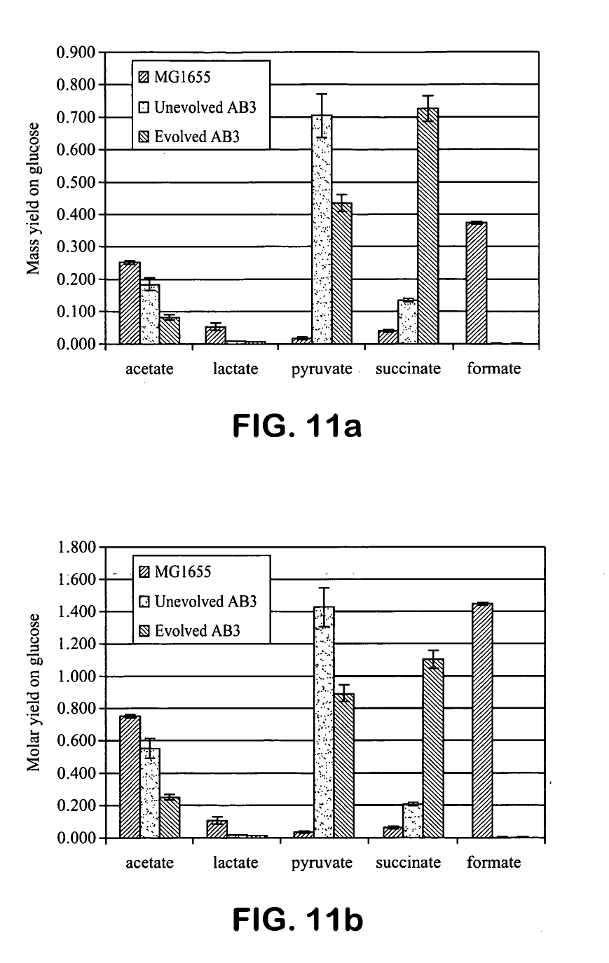

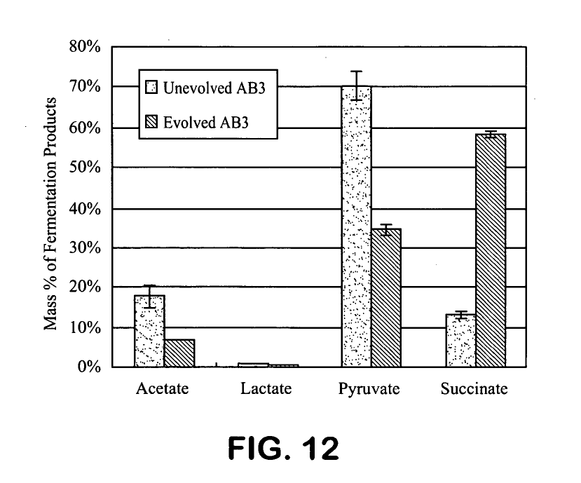

Industrial fermentations are rarely carried out under completely anaerobic conditions. Therefore, the evolved AB3 strain was further characterized under microaerobic growth. The product profile under these growth conditions is shown in FIG. 11 and reveals a shift towards higher succinate formation when compared to its unevolved parent. FIG. 12 shows that the mass percentage of succinate in the fermentation products increased more than four-fold accompanied by approximately 50% and 40% decreases in pyruvate and acetate, respectively. The final molar yield of succinate was 1.1 mol/mol glucose which was very similar to the 1.2 mol/mol glucose yield predicted by the model for this evolved succinate strain (see above description and FIG. 3, rightmost boundary of mutant strain). In contrast to the sample taken directly from the Evolugator, there was still significant pyruvate accumulation, though less than with unevolved AB3 strain. This result indicates that there is likely more 02 present in the machine than in the microaerobic shake flasks. Further evolution time, particularly under improved anaerobic conditions, can be employed, for example, to reduce or eliminate the pyruvate bottleneck altogether. The in silico predictions point at a fermentation profile of 2 moles succinate to I mole acetate after the organism is optimized for cell growth, and the strain characterization data further cooberrate that the expected strain performance can be achieved.

Conclusions

This Example shows the construction and performance characterization of two Escherichia coli strains described in Example I which were designed in silico by the OptKnock computational framework for the growth-coupled production of succinate. The first strain, named AB3, included deletions in adhE, pflA, and ldhA, while the second strain, AB4, included deletions in ackA-pta, dhaKLM, ptsG, pykA, and pykF. These strains, as well as the parent strains AB1 and AB2 created during their construction, exhibited increased succinate yields over the wild-type E. coli MG 1655 strain into which the deletions were introduced. In fact, strain AB4 exhibited nearly a ten-fold increase in succinate yield over the wild-type strain.

The wild-type E. coli strain MG1655 and AB3 also were subjected to adaptive evolution using the Evolugator™ technology to increase their rates of growth. For the wild-type strain, the evolution procedure improved the growth rate nearly 50% under anaerobic conditions but, as expected, did not significantly affect the final product profile. For strain AB3, both the growth rate and succinate yield were increased by the evolution step, while the secretion of the byproduct pyruvate was decreased significantly. This study further coorborates that the OptKnock computational approach can identify combinations of gene deletions that result in the increased production of succinate in E. coli. Furthermore, this study further demonstrated that an adaptive evolution approach can be utilized to drive the performance of an OptKnock designed strain towards the computationally predicted overproduction phenotype.

TABLE 1
Reaction combinations targeted for removal to enhance succinate production
in E. coli. The first seven are mentioned explicitly in the text.

| Succinate* | Biomass^{&} | Metabolic modifications Targeted for Removal^{†,‡,#} | | | | | |
|---|---|---|---|---|---|---|---|
| 8.74 | 0.16 | ADHEr | LDH_D | | | | |
| 9.37 | 0.08 | ADHEr | LDH_D | PTAr | | | |
| 0.84 | 0.19 | PFL | LDH_D | | | | |
| 12.25 | 0.13 | PFL | LDH_D | ADHEr | | | |
| 8.55 | 0.17 | PTAr | PYK | ATPS4r | SUCD1i | DHAPT | |
| 16.10⁺ | 0.08⁺ | PTAr | PYK | GLCpts | DHAPT | | |
| 16.10⁺ | 0.08⁺ | PTAr | PYK | GLCpts | DHAPT | ADHEr | LDH_D |
| 15.06 | 0.05 | ADHEr | G6PDHy | LDH_D | PTAr | PYRt2 | THD2 |
| 14.59 | 0.04 | AKGD | ATPS4r | GLCpts | PTAr | PYK | |
| 14.09 | 0.03 | ADHEr | ATPS4r | FUM | LDH_D | PFL | |
| 13.20 | 0.02 | DHAPT | GLCpts | PDH | PFL | PYK | |
| 13.09 | 0.03 | DHAPT | GLCpts | PYK | TKT2 | | |
| 13.05 | 0.03 | DHAPT | GLCpts | PYK | | | |
| 12.72 | 0.12 | ADHEr | CYTBD | CYTBO3 | LDH_D | PFL | THD2 |
| 10.51 | 0.12 | ATPS4r | GLCpts | PDH | | | |
| 10.31 | 0.12 | ADHEr | ATPS4r | GLCpts | | | |
| 9.90 | 0.13 | ADHEr | GLCpts | THD2 | | | |
| 9.90 | 0.13 | ADHEr | CYTBD | CYTBO3 | GLCpts | THD2 | |
| 9.88 | 0.15 | ADHEr | LDH_D | THD2 | | | |
| 9.83 | 0.02 | GLCpts | PGI | PYK | | | |
| 9.83 | 0.02 | GLCpts | PYK | TPI | | | |
| 9.81 | 0.02 | ADHEr | GLCpts | PGI | | | |
| 9.77 | 0.02 | ADHEr | GLCpts | TPI | | | |
| 9.75 | 0.03 | ATPS4r | FUM | PTAr | TPI | | |
| 9.73 | 0.03 | ATPS4r | PTAr | SUCD1i | TPI | | |
| 9.68 | 0.03 | AKGD | ATPS4r | PTAr | TPI | | |
| 9.67 | 0.03 | ATPS4r | PTAr | SUCOAS | TPI | | |
| 9.33 | 0.09 | GLCpts | PTAr | PYK | | | |
| 9.32 | 0.08 | LDH_D | PDH | PFL | | | |
| 9.18 | 0.13 | ADHEr | GLCpts | TKT2 | | | |
| 9.13 | 0.11 | ADHEr | FUM | LDH_D | | | |
| 9.13 | 0.11 | ADHEr | LDH_D | MDH | | | |
| 9.12 | 0.12 | GLCpts | PFL | PYK | | | |
| 9.12 | 0.11 | ADHEr | FRD3 | GLCpts | | | |
| 9.12 | 0.11 | ADHEr | GLCpts | NADH8 | | | |
| 9.10 | 0.13 | ADHEr | GLCpts | RPE | | | |
| 9.07 | 0.13 | ATPS4r | GLCpts | PYK | | | |
| 9.03 | 0.13 | ADHEr | GLCpts | TAL | | | |
| 9.03 | 0.14 | GLCpts | ME2 | PYK | | | |
| 9.02 | 0.14 | ADHEr | GLCpts | TKT1 | | | |
| 9.01 | 0.16 | ADHEr | LDH_D | TKT2 | | | |
| 9.00 | 0.13 | ADHEr | CBMK2 | GLCpts | | | |
| 8.99 | 0.14 | ADHEr | GLCpts | | | | |
| 8.98 | 0.14 | ADHEr | ATPS4r | THD2 | | | |
| 8.96 | 0.14 | ADHEr | FBA | LDH_D | | | |
| 8.96 | 0.14 | ADHEr | LDH_D | PFK | | | |
| 8.92 | 0.16 | ADHEr | LDH_D | RPE | | | |
| 8.90 | 0.16 | ADHEr | ATPS4r | LDH_D | | | |
| 8.89 | 0.14 | ADHEr | FRD3 | LDH_D | | | |
| 8.89 | 0.14 | ADHEr | LDH_D | NADH8 | | | |
| 8.89 | 0.16 | ATPS4r | FUM | MTHFD | PTAr | | |
| 8.85 | 0.16 | ATPS4r | MTHFD | PTAr | SUCD1i | | |
| 8.83 | 0.16 | ADHEr | LDH_D | TAL | | | |
| 8.83 | 0.14 | ADHEr | CBMK2 | FRD3 | | | |
| 8.83 | 0.14 | ADHEr | CBMK2 | NADH8 | | | |
| 8.83 | 0.16 | ATPS4r | FTHFD | FUM | PTAr | | |
| 8.82 | 0.16 | ADHEr | LDH_D | TKT1 | | | |
| 8.81 | 0.14 | ADHEr | FRD3 | | | | |
| 8.81 | 0.14 | ADHEr | NADH8 | | | | |
| 8.77 | 0.13 | DHAPT | PTAr | PYK | | | |
| 8.76 | 0.15 | ADHEr | GLUDy | | | | |
| 8.76 | 0.16 | ADHEr | CBMK2 | LDH_D | | | |
| 8.70 | 0.15 | DHAPT | PFL | PYK | | | |
| 8.67 | 0.16 | ADHEr | CBMK2 | PIt6 | | | |
| 8.66 | 0.16 | ADHEr | CBMK2 | | | | |
| 8.66 | 0.16 | ADHEr | PIt6 | | | | |
| 8.64 | 0.16 | ADHEr | | | | | |
| 8.64 | 0.17 | ATPS4r | PTAr | SUCD1i | | | |
| 8.37 | 0.17 | AKGD | ATPS4r | PTAr | | | |
| 8.32 | 0.15 | ADHEr | CYTBD | CYTBO3 | THD2 | | |
| 8.31 | 0.18 | ATPS4r | PTAr | SUCOAS | | | |
| 7.42 | 0.14 | GLCpts | PYK | TKT2 | | | |
| 7.23 | 0.14 | GLCpts | PYK | RPE | | | |
| 7.22 | 0.12 | FRD3 | GLCpts | PYK | | | |
| 7.22 | 0.12 | GLCpts | NADH8 | PYK | | | |
| 7.13 | 0.12 | ATPS4r | FBA | PDH | | | |
| 7.13 | 0.12 | ATPS4r | PDH | PFK | | | |
| 7.07 | 0.14 | GLCpts | PYK | TAL | | | |
| 7.05 | 0.14 | GLCpts | PYK | TKT1 | | | |
| 6.91 | 0.03 | ATPS4r | PDH | TPI | | | |
| 6.88 | 0.13 | CBMK2 | GLCpts | PYK | | | |
| 6.86 | 0.14 | GLCpts | PIt6 | PYK | | | |
| 6.83 | 0.14 | GLCpts | PYK | | | | |
| 6.83 | 0.14 | CYTBD | CYTBO3 | GLCpts | PYK | | |
| 6.52 | 0.17 | DHAPT | PYK | TKT2 | | | |
| 6.34 | 0.17 | DHAPT | PYK | RPE | | | |
| 6.29 | 0.15 | DHAPT | FRD3 | PYK | | | |
| 6.29 | 0.15 | DHAPT | NADH8 | PYK | | | |
| 6.17 | 0.17 | DHAPT | PYK | TAL | | | |
| 6.16 | 0.17 | ATPS4r | DHAPT | PYK | | | |
| 6.15 | 0.17 | DHAPT | PYK | TKT1 | | | |
| 6.10 | 0.17 | ATPS4r | PDH | TKT1 | | | |
| 6.10 | 0.17 | ATPS4r | PDH | TAL | | | |
| 6.05 | 0.17 | ATPS4r | G6PDHy | PDH | | | |
| 6.05 | 0.17 | ATPS4r | PDH | PGDH | | | |
| 6.04 | 0.17 | ATPS4r | PDH | RPE | | | |
| 5.99 | 0.16 | CBMK2 | DHAPT | PYK | | | |
| 5.97 | 0.17 | ATPS4r | PDH | TKT2 | | | |
| 5.97 | 0.17 | DHAPT | PIt6 | PYK | | | |
| 5.94 | 0.17 | DHAPT | PYK | | | | |
| 5.94 | 0.17 | CYTBD | CYTBO3 | DHAPT | PYK | | |
| 5.74 | 0.59 | MTHFD | PGDH | SUCD1i | THD2 | | |
| 5.64 | 0.60 | MTHFD | SUCD1i | THD2 | TKT1 | | |
| 5.62 | 0.60 | MTHFD | SUCD1i | TAL | THD2 | | |
| 5.51 | 0.61 | MTHFD | RPE | SUCD1i | THD2 | | |
| 5.50 | 0.62 | MTHFD | PTAr | SUCD1i | TAL | | |
| 5.43 | 0.61 | MTHFD | PGDH | PTAr | SUCD1i | | |
| 5.40 | 0.63 | MTHFD | PTAr | RPE | SUCD1i | | |
| 5.38 | 0.62 | MTHFD | SUCD1i | THD2 | TKT2 | | |
| 5.38 | 0.18 | ATPS4r | GLYCLTt2r | PDH | | | |
| 5.36 | 0.18 | ATPS4r | PDH | | | | |
| 5.33 | 0.62 | MTHFD | PTAr | SUCD1i | TKT1 | | |
| 2.34 | 0.03 | ATPS4r | PTAr | TP1 | | | |
| 2.26 | 0.14 | G6PDHy | PDH | PTAr | | | |
| 2.26 | 0.14 | PDH | PGDH | PTAr | | | |
| 2.11 | 0.13 | PDH | PFL | THD2 | | | |
| 1.97 | 0.15 | PDH | PTAr | TKT1 | | | |
| 1.95 | 0.15 | PDH | PTAr | TAL | | | |
| 1.79 | 0.15 | PDH | PTAr | RPE | | | |
| 1.75 | 0.17 | ATPS4r | FUM | PTAr | | | |
| 1.73 | 0.17 | ATPS4r | PTAr | THD2 | | | |
| 1.72 | 0.17 | ATPS4r | PTAr | TKT2 | | | |
| 1.69 | 0.16 | ATPS4r | MTHFD | PTAr | | | |
| 1.68 | 0.17 | ATPS4r | PTAr | RPE | | | |
| 1.67 | 0.17 | ATPS4r | PTAr | PTAr | | | |
| 1.65 | 0.17 | ATPS4r | PTAr | TAL | | | |
| 1.65 | 0.17 | ATPS4r | MDH | PTAr | | | |
| 1.65 | 0.17 | ATPS4r | PTAr | TKT1 | | | |
| 1.64 | 0.17 | ATPS4r | PIt6 | PTAr | | | |
| 1.62 | 0.17 | ATPS4r | CBMK2 | PTAr | | | |
| 1.61 | 0.15 | PDH | PTAr | TKT2 | | | |
| 1.59 | 0.17 | ATPS4r | PTAr | | | | |
| 1.56 | 0.18 | GLYCL | PFL | TKT2 | | | |
| 1.40 | 0.18 | G6PDHy | ME2 | THD2 | | | |
| 1.35 | 0.18 | G6PDHy | MDH | THD2 | | | |
| 1.33 | 0.19 | GLYCL | PFL | RPE | | | |
| 1.31 | 0.17 | PFL | THD2 | TKT2 | | | |
| 1.24 | 0.19 | PFL | RPE | | | | |
| 1.24 | 0.19 | PFL | PTAr | TAL | | | |
| 1.22 | 0.19 | PFL | PTAr | TKT1 | | | |
| 1.12 | 0.19 | GLYCL | PFL | TAL | | | |
| 1.09 | 0.19 | GLYCL | PFL | TKT1 | | | |
| 1.08 | 0.17 | PFL | RPE | THD2 | | | |
| 1.05 | 0.14 | PDH | PFL | TKT2 | | | |
| 1.05 | 0.19 | PFL | PTAr | | | | |
| 1.04 | 0.19 | HSK | PFL | | | | |
| 1.04 | 0.19 | PFL | THRS | | | | |
| 1.04 | 0.19 | PFL | TAL | | | | |
| 1.01 | 0.19 | PFL | TKT1 | | | | |
| 0.92 | 0.84 | FUM | ORNDC | SUCFUMtdc | | | |
| 0.89 | 0.85 | FUM | SUCFUMtdc | | | | |
| 0.88 | 0.17 | PFL | TAL | THD2 | | | |
| 0.87 | 0.14 | PDH | PFL | RPE | | | |
| 0.87 | 0.19 | GART | PFL | | | | |
| 0.86 | 0.19 | GLXCBL | GLYCLTt2r | PFL | | | |
| 0.86 | 0.19 | GLYCK | GLYCLTt2r | PFL | | | |
| 0.86 | 0.17 | PFL | PTAr | THD2 | | | |
| 0.86 | 0.17 | PFL | THD2 | TKT1 | | | |
| 0.84 | 0.19 | PFL | | | | | |
| 0.81 | 0.14 | PDH | PFL | PTAr | | | |
| 0.81 | 0.82 | MTHFD | PFL | SUCD1i | | | |
| 0.75 | 0.71 | ENO | GAPD | SUCD1i | | | |
| 0.75 | 0.71 | ENO | PGK | SUCD1i | | | |
| 0.75 | 0.71 | GAPD | PGM | SUCD1i | | | |
| 0.75 | 0.71 | PGK | PGM | SUCD1i | | | |
| 0.71 | 0.14 | PDH | PFL | TAL | | | |
| 0.69 | 0.14 | PDH | PFL | TKT1 | | | |
| 0.67 | 0.64 | FUM | NADH6 | SUCD1i | | | |
| 0.67 | 0.17 | PFL | THD2 | | | | |
| 0.66 | 0.14 | PDH | PFL | | | | |
| 0.47 | 0.19 | MTHFD | PFL | TKT2 | | | |
| 0.44 | 0.87 | ACCOAL | SUCD1i | SUCOAS | | | |
| 0.44 | 0.87 | PPCSCT | SUCD1i | SUCOAS | | | |
| 0.43 | 0.19 | PFL | PTAr | TKT2 | | | |
| 0.42 | 0.19 | GARFT | PFL | | | | |
| 0.37 | 0.35 | ATPS4r | FUM | | | | |
| 0.31 | 0.19 | HSK | MTHFD | | | | |
| 0.31 | 0.19 | MTHFD | THRS | | | | |
| 0.28 | 0.88 | DHORD2 | SUCD1i | | | | |
| 0.23 | 0.19 | MTHFD | PFL | RPE | | | |
| 0.23 | 0.22 | CYTBD | CYTBO3 | FUM | | | |
| 0.23 | 0.22 | FUM | | | | | |
| 0.23 | 0.22 | MDH | ME2 | | | | |
| 0.22 | 0.19 | PFL | TKT2 | | | | |
| 0.21 | 0.20 | ATPS4r | MDH | | | | |
| 0.20 | 0.19 | MDH | PFL | | | | |
| 0.19 | 0.19 | PFL | PTAr | RPE | | | |
| 0.17 | 0.21 | ACCOAL | SUCOAS | | | | |
| 0.17 | 0.21 | PPCSCT | SUCOAS | | | | |
| 0.16 | 0.15 | MDH | PDH | PTAr | | | |
| 0.14 | 0.21 | FBA | MDH | | | | |
| 0.14 | 0.21 | MDH | PFK | | | | |
| 0.13 | 0.21 | FTHFD | HSK | | | | |
| 0.13 | 0.21 | FTHFD | THRS | | | | |

*Predicted mmol of succinate per 10 mmol of glucose
^{&}Predicted gDW biomass per 10 mmol glucose
^{†}Reactions can be removed by eliminating the appropriate gene(s) shown in Supplementary Table 2.
^{‡}ACKr, F6PA, MTHFC, PGL, and SUCD4 can be removed along with or as an alternative to PTAr, DHAPT, MTHFD, G6PDHy, and SUCD1i, respectively.
^{#}Any combination (i.e., at least one and at most all) of the listed reaction deletions could conceivably have the desired effect.
⁺Solution assumes no non-growth associated maintenance requirement. All others simulations assume a non-growth associated maintenance requirement of 7.6 mmol/gDW/hr.

TABLE 2
Known E. coli genes responsible for catalyzing the reactions targeted for removal

| Reaction | | Genes Encoding the Enzyme(s) Catalyzing |
|---|---|---|
| Abbreviation | Reaction Stoichiomelry* | Each Reaction^{&†} |
| ACCOAL | [c]: atp + coa + ppa −−> adp + pi + ppcoa | b0335 |
| ACKr | [c]: ac + atp <==> actp + adp | b2296 (ackA), b1849, b3115 |
| ADHEr | [c]: accoa + (2) h + (2) nadh <==> coa + etoh + (2) nad | B1241 (adhE) |
| AKGD | [c]: akg + coa + nad −−> co2 + nadh + succoa | b0116, b0726, b0727 |
| ATPS4r | adp[c] + (4) h[e] + pi[c] <==> atp[c] + (3) h[c] + h2o[c] | b3736 (atp), b3737, b3738, b373l , b3732, |
| | | b3733, b3734, b3735, b3739 |
| CBMK2 | [c]: atp + co2 + nh4 −−> adp + cbp + (2) h | b052l, b2874, b0323 |
| CYTBD | (2) h[c] + (0.5) o2[c] + ubg8h2[c] −−> (2) h[e] + h2o[c] + ubq8[c] | b0733, b0734 |
| CYTBO3 | (2.5) h[c] + (0.5) o2[c] + ubq8h2[c] −−> (2.5) h[e]+ h2o[c] + ubq8[c] | b0429, b0430, b0431, b0432 |
| DHAPT | [c]: dha + pep −−> dhap + pyr | b1200 (dhaM), b1199 (dhal), b1198 |
| | | (dhaK), b2415, b2416 |
| DHORD2 | [c]: dhor-S + ubq8 −−> orot + ubq8h2 | b0945 |
| D-LACt2 | h[e] + lac-D[e] <==> h[c] + lac-D[c] | b3603, b2975 |
| ENO | [c]: 2pg <==> h2o + pep | b2779 |
| F6PA | [c]: f6p <==> dha + g3p | b0825, b3946 |
| FBA | [c]: fdp <==> dhap + g3p | b2925, b2097, b1773 |
| FORt | for[e] <==> for[c] | b0904, b2492 |
| FRD3 | [c]: 2dmmql8 + fum −−> 2dmmq8 + succ | b4151, b4152, b4153, b4154 |
| FTHFD | [c]: 10fthf + h2o −−> for + h + thf | b1232 |
| FUM | [c]: fum + h2o <==> mal-L | b1612, b4122, b1611 |
| G6PDHy | [c]: g6p + nadp <==> 6pgl + h + nadph | b1852 |
| GAPD | [c]: g3p + nad + pi <==> 13dpg + h + nadh | b1779, b1416, b1417 |
| GARFT | [c]: 10fthf + gar <==> fgam + h + thf | b2500 |
| GART | [c]: atp + for + gar −−> adp + fgam + h + pi | b1849 |
| GLCpts | glc-D[e] + pep[c] −−> g6p[c] + pyr[c] | b2417 (ptsG), b1101, b2415, b2416, |
| | | b2417, b1621, b2415, b2416, b1817, |
| | | b1818, b1819, b2415, b2416 |
| GLUDy | [c]: glu-L + h2o + nadp <==> akg + h + nadph + nh4 | b1761 |
| GLXCBL | [c]: (2) glx + h −−> 2h3opp + co2 | b0507 |
| GLYCK | [c]: atp + glyc-R −−> 3pg + adp + h | b0514, b3124 |
| GLYCL | [c]: gly + nad + thf −−> co2 + mlthf + nadh + nh4 | b2904, b2903, b2905, b0116 |
| GLYCLTt2r | glyclt[e] + h[e] <==> glyclt[c] + h[c] | b3603, b2975 |
| HSK | [c]: atp + hom-L −−> adp + h + phom | b0003 |
| LDH_D | [c]: lac-D + nad <==> h + nadh + pyr | b2133, b1380 (ldhA) |
| MDH | [c]: mal-L + nad <==> h + nadh + oaa | b3236 |
| ME2 | [c]: mal-L + nadp −−> co2 + nadph + pyr | b2463 |
| MTHFC | [c]: h2o + methf <==> 10fthf + h | b0529 |
| MTHFD | [c]: mlthf + nadp <==> methf + nadph | b0529 |
| NADH6 | (4.5) h[c] + nadh[c] + ubq8[c] −−> (3.5) h[e] + nad[c] + ubq8h2[c] | b2276, b2277, b2278, b2279, b2280, |
| | | b2281, b2282, b2283, b2284, b2285, |
| | | b2286, b2287, b2288 |
| NADH8 | 2dmmq8[c] + (3.8) h[c] + nadh[c] −−> 2dmmql8[c] + (2.8) h[e] + nad[c] | b2276, b2277, b2278, b2279, b2280, |
| | | b2281, b2282, b2283, b2284, b2285, |
| | | b2286, b2287, b2288 |
| ORNDC | [c]: h + orn-L −−> co2 + ptrc | b2965, b0693 |
| PDH | [c]: coa + nad + pyr −−> accoa + co2 + nadh | b0114, b0115, b0116 |
| PFK | [c]: atp + f6p −−> adp + fdp + h | b3916, b1723 |
| PFL | [c]: coa + pyr −−> accoa + for | b3951, b3952, b3114, b0902 (pflA), |
| | | b0903 (pflB) |
| PGDH | [c]: 6pgc + nadp −−> co2 + nadph + ru5p-D | b2029 |
| PGI | [c]: g6p <==> f6p | b4025 |
| PGK | [c]: 13dpg + adp <==> 3pg + atp | b2926, b0755, b4395 |
| PGL | [c]: 6pgl + h2o −−> 6pgc + h | not known |
| PGM | [c]: 3pg <==> 2pg | b3612 |
| Plt6 | h[e] + pi[e] <==> h[c] + pi[c] | b3493, b2987 |
| PPCSCT | [c]: ppcoa + succ −−> ppa + succoa | b2920 |
| PTAr | [c]: accoa + pi <==> actp + coa | b2297 (pta), b2458 |
| PYK | [c]: adp + h + pep −−> atp + pyr | b1854 (pykA), b1676, (pykF) |
| PYRt2 | h[e] + pyr[e] <==> h[c] + pyr[c] | not known |
| RPE | [c]: ru5p-D <==> xu5p-D | b3386, b4301 |
| SUCDli | [c]: fad + succ −−> fadh2 + fum | b0721 (sdhA), b0722 (sdhB), b0723 |
| | | (sdhC), b0724 sdhC) |
| SUCD4 | [c]: fadh2 + ubq8 <==> fad + ubg8h2 | b0721 (sdhA), b0722 (sdhB), b0723 |
| | | (sdhC), b0724 sdhC) |
| SUCFUMtdc | fum[e] + succ(c) <==> fum[c] + succ[e] | b4138, b4123, b0621 |
| SUCOAS | [c]: atp + coa + succ <==> adp + pi + succoa | b0728, b0729 |
| TAL | [c]: g3p + s7p <==> e4p + f6p | b0008, b2464 |
| THD2 | (2) h[e] + nadh[c] + nadp[c] −−> (2) h[c] + nad[c] + nadph[c] | b1602, b1603 |
| THRS | [c]: h2o + phom −−> pi + thr-L | b0004 |
| TKT1 | [c]: r5p + xu5p-D <==> g3p + s7p | b2935, b2465 |
| TKT2 | [c]: e4p + xu5p-D <==> f6p + g3p | b2935, b2465 |
| TPI | [c]: dhap <==> g3p | b3919 |

*Metabolite names corresponding to abbreviations are found in Supplementary Table 3.
^{&}OptKnock identifies reactions to be eliminated from an organism to enhance biochemical production. Any combination (i.e., at least one and at most all) of the listed gene deletions could conceivably have the desired effect of ensuring that the corresponding reaction is non-functional in E. coli. The most practical experimental strategy for eliminating the reactions targeted for removal must be determined on a case-by-case basis.
†Common gene names used in the text are given in parentheses following the corresponding numeric gene name.

TABLE 3
Metabolite names corresponding to abbreviations from Supplementary Table 2

| Metabolite | | |
|---|---|---|
| Abbreviation | Compartment | Metabolite Name |
| 10fthf | Cytosol | 10-Formyltetrahydrofolate |
| 13dpg | Cytosol | 3-Phospho-D-glycerqyl phosphate |
| 2dmmq8 | Cytosol | 2-Demethylmenaquinone 8 |
| 2dmmql8 | Cytosol | 2-Demethylmenaquinol 8 |
| 2h3opp | Cytosol | 2-Hydroxy-3-oxopropanoate |
| 2pg | Cytosol | D-Glycerate 2-phosphate |
| 3pg | Cytosol | 3-Phospho-D-glycerate |
| 6pgc | Cytosol | 6-Phospho-D-gluconate |
| 6pgl | Cytosol | 6-phospho-D-glucono-1,5-lactone |
| ac | Cytosol | Acetate |
| ac[e] | Extra-organism | Acetate |
| accoa | Cytosol | Acetyl-CoA |
| actp | Cytosol | Acetyl phosphate |
| adp | Cytosol | ADP |
| akg | Cytosol | 2-Oxoglutarate |
| atp | Cytosol | ATP |
| cbp | Cytosol | Carbamoyl phosphate |
| co2 | Cytosol | CO2 |
| coa | Cytosol | Coenzyme A |
| dha | Cytosol | Dihydroxyacetone |
| dhap | Cytosol | Dihydroxyacetone phosphate |
| dhor-S | Cytosol | (S)-Dihydroorotate |
| e4p | Cytosol | D-Erythrose 4-phosphate |
| etoh | Cytosol | Ethanol |
| etoh[e] | Extra-organism | Ethanol |
| f6p | Cytosol | D-Fructose 6-phosphate |
| fad | Cytosol | FAD |
| fadh2 | Cytosol | FADH2 |
| fdp | Cytosol | D-Fructose 1,6-bisphosphate |
| fgam | Cytosol | N2-Formyl-N1-(5-phospho-D-ribosyl)glycinamide |
| for | Cytosol | Formate |
| for[e] | Extra-organism | Formate |
| fum | Cytosol | Fumarate |
| fum[e] | Extra-organism | Fumarate |
| g3p | Cytosol | Glyceraldehyde 3-phosphate |
| g6p | Cytosol | D-Glucose 6-phosphate |
| gar | Cytosol | N1-(5-Phospho-D-ribosyl)glycinamide |
| glc-D[e] | Extra-organism | D-Glucose |
| glu-L | Cytosol | L-Glutamate |
| glx | Cytosol | Glyoxylate |
| gly | Cytosol | Glycine |
| glyclt | Cytosol | Glycolate |
| glyclt[e] | Extra-organism | Glycolate |
| glyc-R | Cytosol | (R)-Glycerate |
| h | Cytosol | H+ |
| h[e] | Extra-organism | H+ |
| h2o | Cytosol | H2O |
| hom-L | Cytosol | L-Homoserine |
| lac-D | Cytosol | D-Lactate |
| lac-D[e] | Extra-organism | D-Lactate |
| mal-L | Cytosol | L-Malate |
| methf | Cytosol | 5,10-Methenyltetrahydrofolate |
| mlthf | Cytosol | 5,10-Methylenetetrahydrofolate |
| nad | Cytosol | Nicotinamide adenine dinucleotide |
| nadh | Cytosol | Nicotinamide adenine dinucleotide - reduced |
| nadp | Cytosol | Nicotinamide adenine dinucleotide phosphate |
| nadph | Cytosol | Nicotinamide adenine dinucleotide phosphate - reduced |
| nh4 | Cytosol | Ammonium |
| o2 | Cytosol | O2 |
| oaa | Cytosol | Oxaloacetate |
| orn-L | Cytosol | L-Ornithine |
| orot | Cytosol | Orotate |
| pep | Cytosol | Phosphoenolpyruvate |
| phom | Cytosol | O-Phospho-L-homoserine |
| pi | Cytosol | Phosphate |
| pi[e] | Extra-organism | Phosphate |
| ppa | Cytosol | Propionate |
| ppcoa | Cytosol | Propanoyl-CoA |
| ptrc | Cytosol | Putrescine |
| pyr | Cytosol | Pyruvate |
| pyr[e] | Extra-organism | Pyruvate |
| r5p | Cytosol | alpha-D-Ribose 5-phosphate |
| ru5p-D | Cytosol | D-Ribulose 5-phosphate |
| s7p | Cytosol | Sedoheptulose 7-phosphate |
| succ | Cytosol | Succinate |
| succ[e] | Extra-organism | Succinate |
| succoa | Cytosol | Succinyl-CoA |
| thf | Cytosol | 5,6,7,8-Tetrahydrofolate |
| thr-L | Cytosol | L-Threonine |
| ubq8 | Cytosol | Ubiquinone-8 |
| ubq8h2 | Cytosol | Ubiquinol-8 |
| xu5p-D | Cytosol | D-Xylulose 5-phosphate |

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A non-naturally occurring microorganism comprising one or more gene disruptions, said one or more gene disruptions occurring in genes encoding an enzyme obligatory coupling succinate production to growth of said microorganism when said gene disruption reduces an activity of said enzyme, whereby said one or more gene disruptions confers stable growth-coupled production of succinate onto said non-naturally occurring microorganism.

2. The non-naturally occurring microorganism of claim 1, wherein said one or more gene disruptions comprise a metabolic modification listed in Table 1.

3. The non-naturally occurring microorganism of claim 1, wherein said one or more gene disruptions comprise a deletion of said one or more genes.

4. The non-naturally occurring microorganism of claim 1, wherein said non-naturally occurring microorganism is selected from the group of microorganisms having a metabolic modification listed in Table 1.

5. The non-naturally occurring microorganism of claim 1, wherein said microorganism comprises a bacterium, yeast or fungus.

6. The non-naturally occurring microorganism of claim 5, wherein said bacteria comprises a species selected from E. coli, A. succiniciproducens, A. succinogenes, M. succiniciproducens, R. etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonasfluorescens, and Pseudomonas putida.

7. The non-naturally occurring microorganism of claim 5, wherein said yeast comprises a species selected from Saccharomyces cerevisiae, Schizosaccharomyces pombe, Kluyveromyces lactis, Kluyveromyces marxianus, Aspergillus terreus, Aspergillus niger, and Pichia pastoris.

8. A non-naturally occurring microorganism comprising a set of metabolic modifications obligatory coupling succinate production to growth of said microorganism, said set of metabolic modifications comprising disruption of one or more genes selected from the set of genes comprising:
(a) adhE, ldhA
(b) adhE, ldhA, acka-pta
(c)pfl, ldhA
(d) pfl, ldhA, adhE
(e) ackA-pta, pykF, atpF, sdhA
(f) ackA-pta, pykf, ptsG, or
(g) ackA-pta, pykF, ptsG, adhE, ldhA,
or an ortholog thereof, wherein said microorganism exhibits stable growth-coupled production of succinate.

9. The non-naturally occurring microorganism of claim 8, wherein said genes encoding said metabolic modification (e) ackA-pta, pykf, atpF, sdhA further comprises disruption of at least one gene selected from pyka, atpH, sdhB or dhaKLM.

10. The non-naturally occurring microorganism of claim 8, wherein said genes encoding said metabolic modification (f) ackA-pta, pykF, ptsG further comprises disruption of at least one gene selected from pykA or dhaKLM.

11. The non-naturally occurring microorganism of claim 8, wherein said genes encoding said metabolic modification (g) ackA-pta, pykF, ptsG, adhE, ldhA further comprises disruption of at least one gene selected from pykA or dhaKLM.

12. The non-naturally occurring microorganism of claim 8, wherein said disruption of one or more genes comprises a deletion.

13. The non-naturally occurring microorganism of claim 8, wherein said microorganism comprises a bacterium, yeast or fungus.

14. The non-naturally occurring microorganism of claim 13, wherein said bacteria comprises a species selected from E. coli, A. succiniciproducens, A. succinogenes, M. succiniciproducens, R. etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicuin, Pseutdomonasfluorescens, and Pseudomonas putida.

15. The non-naturally occurring microorganism of claim 13, wherein said yeast comprises a species selected from Saccharomyces cerevisiae, Schizosaccharomyces pombe, Kluyveromyces lactis, Kluyveromyces marxianus, Aspergillus terreus, Aspergillus niger, and Pichia pastoris.

16. A method of producing a non-naturally occurring microorganism having stable growth-coupled production of succinate, comprising:
(a) identifying in silico a set of metabolic modifications requiring succinate production during exponential growth, and
(b) genetically modifying a microorganism to contain said set of metabolic modifications requiring succinate production.

17. The-method of claim 16, wherein said set of metabolic modifications are identified by an in silico method selected from OptKnock or SimPheny.

18. The method of claim 16, wherein said set of metabolic modifications comprise functional disruption of one or more metabolic reactions.

19. The method of claim 18, wherein said metabolic modifications are disrupted by gene deletion.

20. The method of claim 18, wherein said non-naturally occurring microorganism comprises a microorganism having a metabolic modification selected from the set of metabolic modifications listed in Table 1.

21. The method of clam 16, further comprising culturing said genetically modified microorganism.

22. The method of claim 21, further comprising adaptively evolving said genetically modified microorganism under conditions requiring succinate production.

23. The method of claim 16, wherein said non-naturally occurring microorganism comprises a bacterium, yeast or fungus.

24. The method of claim 23, wherein said bacteria comprises a species selected from E. coli, A. succiniciproducens, A. succinogenes, M. succiniciproducens, R. etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida.

25. The non-naturally occurring microorganism of claim 23, wherein said yeast comprises a species selected from Saccharomyces cerevisiae, Schizosaccharomyces pombe, Kluyveromyces lactis, Kluyveromyces marxianus, Aspergillus terreus, Aspergillus niger, and Pichia pastoris.

26. A microorganism produced by the method of claim 16 or 22.

27. A method of producing succinate coupled to the growth of a microorganism, comprising:
(a) culturing under exponential growth phase in a sufficient amount of neutrients and media a non-naturally occurring microorganism comprising a set of metabolic modifications obligatory coupling succinate production to growth of said microorganism, said set of metabolic modifications comprising disruption of one or more genes selected from the set of genes comprising:
(1) adhE, ldhA
(2) adhE, ldhA, ackA-pta
(3) pfI, ldhA
(4) pfi, ldhA, adhE
(5) ackA-pta, pykF, atpF, sdhA
(6) ackA-pta, pykF, ptsG, or
(7) ackA-pta, pykF, ptsG, adhE, ldhA,
or an ortholog thereof, wherein said microorganism exhibits stable growth-coupled production of succinate, and
(b) isolating succinate produced from said non-naturally occurring microorganism.

28. The method of claim 27, wherein said genes encoding said metabolic modification (5) ackA-pta, pykF, atpF, sdhA further comprises disruption of at least one gene selected from pyka, atpH, sdhB or dhaKLM.

29. The method of claim 27, wherein said genes encoding said metabolic modification (6) ackA-pta, pykF, ptsG further comprises disruption of at least one gene selected from pyka or dhaKLM.

30. The method of claim 27, wherein said genes encoding said metabolic modification (7) acka-pta, pykF, ptsG, adhE, ldhA further comprises disruption of at least one gene selected from pykA or dhaKLM.
